(19) United States
(12) Patent Application Publication
(10) Pub. No.: US 2023/0090223 A1
(43) Pub. Date: Mar. 23, 2023

(54) NANOPARTICLES FOR USE IN THE TREATMENT AND DIAGNOSIS OF CNS DISORDERS

(71) Applicant: UPPSALA THERAPEUTICS AB, UPPSALA (SE)

(72) Inventors: Oommen Podiyan OOMMEN, TAMPERE (FI); Varghese OOMMEN PODIYAN, UPPSALA (SE)

(21) Appl. No.: 17/802,123

(22) PCT Filed: Feb. 26, 2021
(86) PCT No.: PCT/SE2021/050162

§ 371 Date: Aug. 24, 2022

(30) Foreign Application Priority Data
Feb. 28, 2020 (SE) 2050224-1

(51) Int. Cl. A61K 31/727; A61K 33/242; A61K 33/26; A61K 9/14; A61P 25/04

(52) U.S. Cl. CPC A61K 31/727; A61K 33/242; A61K 33/26; A61K 9/146; A61P 25/04

(57) ABSTRACT

A nanoparticle comprising chemically modified heparin, wherein the heparin has been chemically modified by attaching hydrophobic moieties to functional groups of the heparin, said functional groups being selected from hydroxy groups and carboxy groups, for use in the treatment or diagnosis of a brain disorder.

### FIELD OF THE INVENTION

The present invention relates to nanoparticles for use in the treatment and diagnosis of disorders affecting the central nervous system. More particularly, the present invention relates to heparin-containing nanoparticles having a capacity of crossing the blood-brain barrier, for use in the treatment and diagnosis of disorders affecting the central nervous system.

### BACKGROUND OF THE INVENTION

Herein below, any publication will be indicated by the family name of the first author of the publication, followed by the year of publication, with a detailed reference in the reference list at the end of the description.

The blood—brain barrier (BBB) is responsible for physiologically protecting the brain from exposure to toxins and ill effects. However, this protective function of the BBB presents a key challenge of medicine today, with a need to circumvent it in order to deliver drugs to the brain in various central nervous system (CNS) disorders. Further, as age advances, the chances of the development of CNS disorders proportionately increases and the selective permeability of drugs aggravates the issues related to drug delivery and brain targeting.

The BBB has been found unique in nature and to demonstrate selective permeability. It is a complex system made up of a structurally distinct, continuous endothelial cell layer separating the blood from the extracellular fluid of the brain.

In the BBB, the luminal plasma membrane of the endothelial cells is directed towards the blood, while the abluminal plasma membrane faces the brain (Patel, 2009). The presence of adhesion molecules and tight junctions between endothelial cells (Abbott, 2005) and the low density of pinocytes (Stewart, 2000) are some of the key structural features that make the BBB selectively permeable. Advancements in technological tools and biomedical sciences have led to better understanding of not only the detailed structure of the BBB but also the pathophysiological mechanisms of CNS disorders.

One strategy for paracellular delivery of drug across BBB involves transient opening of BBB using small molecules such as mannitol (Godinho, 2018) or recombinant proteins such as human vascular endothelial growth factor (Lundy, 2019) or targeting tight junction claudin-5 (Neuhaus, 2018).

There has been tremendous research in the field of nanomedicine-based drugs. Nanoparticles penetrate the BBB by binding to serum proteins such as albumin or ApoE that interact with the BBB receptors or transporters. E.g. the surfactant polysorbate 80 (or Tween 80) PLGA nanoparticles loaded with AD drug donepezil were targeted to the brain by binding with ApoE in the blood (Bhavna, 2014). Other mechanisms for brain delivery of nanoparticles involve using targeting ligands decorated on the nanoparticle surface that directly interact with the BBB receptors such as transferrin receptor, glucose receptor, insulin receptor etc., e.g., nanoparticles decorated with small molecules such a glucose (Anraku, 2017) or peptides targeting specific receptors such as transferrin (Markoutsa, 2014), integrin binding arginylglycylaspartic acid (RGD) peptides (Zhang, 2012; Ruan, 2017; Qin, 2007), cell-penetrating peptides (Stalmans, 2015) or apolipoprotein peptide analog (Markoutsa, 2014).

Despite these advances, the distress caused by several CNS-related diseases is increasing and present a huge social and economic burden (Chen, 2012). Only the number of new cases of brain and other nervous system cancers amounted to 6.4 per 100,000 men and women per year and the number of deaths was 4.3 per 100,000 men and women per year as per recent statistics from the American Cancer Society (Siegel, 2019). Drug discoveries continue to be made in plentiful numbers but even though novel therapeutic molecules give good results in the early phase of drug development, they often fail to successfully clear subsequent preclinical and clinical trials, owing in part to their inability to cross the BBB.

Gliomas are the most common primary central nervous system (CNS) tumors and, among these, half of all new diagnoses are represented by glioblastoma multiforme (GBM). GBM is known to be one of the most lethal and untreatable human tumors, with a poor prognosis and a median patient survival of approximately 18 months (Di Carlo, 2019). Surgery and radiotherapy in combination with classical alkylating agents such as temozolomide offer little hope to escape a poor prognosis. For these reasons, enormous efforts are currently devoted to refining in vivo and in vitro models with the specific goal of finding new molecular aberrant pathways, suitable to be targeted by a variety of therapeutic approaches, including novel pharmaceutical formulations and immunotherapy strategies.

Extensive surgical resection, though representing the most effective way to increase survival of GBM patients, is hardly feasible depending on tumor localization and infiltration, particularly when highly specialized brain areas are involved, such as those involved in the control of speech, motor function and senses. However, the highly infiltrative behavior of GBM makes surgery ineffective, since tumor cells and glioblastoma stem cells (GSC) colonize the surrounding brain tissue causing relapses located even at distant brain sites (Davis, 2016). Nevertheless, cutting-edge imaging techniques in both diagnostic and surgical phases now make possible a more aggressive surgical approach with limited side effects for patients (Davis, 2016). The imaging techniques include functional magnetic resonance imaging (MRI) and diffusion tensor imaging (DTI), during preparatory phase, and ultrasound, computed tomography (CT) and MRI during surgery.

To overcome the limitation associated with small hydrophobic drugs, various nanoparticle systems have been designed, including liposome vesicles and inorganic nanoparticles.

Liposome vesicles are very flexible tools as they can be loaded by a variety of drugs and are highly biocompatible. Paclitaxel is a classical and effective chemotherapeutic drug that is unable to cross the BBB and for this reason cannot be used for brain tumor treatment. In the aim to make paclitaxel cross the BBB, liposomes have been loaded with the drug and used for GBM therapy (Belhadj, 2017). Also other drugs, such as peptides, monoclonal antibodies, siRNA and other molecules, which otherwise would not be able to pass the BBB, are under investigation for use in brain tumor treatment as liposome formulations.

Inorganic nanoparticles can be used both for diagnostic and therapeutic purposes. Magnetic nanoparticles are the most common nanoparticles used in biomedical applications due to their high biocompatibility; injectable superparamagnetic iron oxide nanoparticles (SPIONs) can also be used in MRI with lower toxicity effects and higher sensitivity compared to conventional contrast agents (Zhang, 2012). In addition, SPIONs display interesting features such as high intratumoral penetration and controlled heating: in fact, besides destroying tumor cells, hyperthermia can also be used to deliver drugs to tumors such as GBM. The BBB permeability of SPIONs could be achieved by decorating these particles with integrin binding RGD peptides (Zhang, 2012). Similarly, gold nanoparticles can be used both as contrast agent for MRI and for photothermal therapy (Pourgholi, 2016).

Low molecular weight heparin (LMWH) and heparin-derived oligosaccharide(s) (HDO) have been clinically used for the management of neurological disorders, such as stroke and Alzheimer's disease.

In vitro cancer studies indicate both heparin and LMWH to inhibit angiogenesis, invasion, and metastasis of solid tumors (Zacharski, 2008). In animal models for different, non-GBM tumors, heparin and LMWH (Mousa, 2009) were shown to inhibit tumor growth, prevent metastasis and prolong survival (Borsig, 2010). The non-anticoagulant activity of heparin on metastasis includes the ability to inhibit cell-cell-interaction through blocking of P- and L-selectin, to inhibit heparanase activity and to inhibit angiogenesis (Borsig, 2010).

Significant intracranial hemorrhage occurs in 20% to 50% of patients with metastatic brain tumors. LMWH is currently used in clinic to treat cancer-related venous thromboembolism (van der Wall, 2017). Administration of LMWH to patients with primary brain tumor or secondary metastatic brain tumor is found to be beneficial to mitigate the risk of intracranial haemorrhage with acceptable risk of bleeding (Lin, 2018). A cohort study of 293 patients with brain metastases treated with LMWH (enoxaparin) reveal that using LWMH does not increase the risk for intracranial haemorrhage and however, there was no difference in the overall survival (Donato, 2015). Although, several studies report the beneficial anti-cancer effects of heparin and LMWH in animal models, there are no reports on the anti-tumor effect of heparins in GBM models. This is probably due to the poor blood-brain barrier permeability of large unfractionated heparin molecules. The ultralow-molecular weight heparin or heparin-derived oligosaccharides C3, on the other hand display efficient BBB permeability (Ma, 2002). Recently, a heparin derived nanocarrier was designed using integrin binding RGD peptide (well known to promote BBB permeability) (Zhang, 2012; Qin, 2007) and hydrophobic anti-angiogenic peptide targeting EphA2 tyrosine kinase receptor (overexpressed on GBM and tumor vasculature) that formed self-assembled particles in blood and suppressed glioma in orthotopic mouse models (Wang, 2016).

Mei et al., (Mei, 2016) have developed heparin-based micelles by conjugating hydrophobic deoxycholate molecules which were subsequently loaded with doxorubicin (DOX). These DOX loaded micelles displayed inhibitory effect on the metastasis and angiogenesis and efficiently suppressed tumor in melanoma animal model in C57BL/6 mouse. The unloaded heparin particles did not exhibit any anti-tumor effects. Cho et al., (Cho, 2008) on the other hand have reported a modest 34% reduction in tumor volume when heparin-deoxycholate conjugate was injected intravenously at 10 mg/kg in athymic BALB/c-nu/nu female nude mice bearing KB tumors (human epidermoid carcinoma cells).

Kuo et al., (Kuo, 2017)) developed heparinized cationic solid lipid nanoparticles that were suggested to be useful to deliver neurotropic growth factor which is a potent growth factor necessary for regeneration of neurons.

Yang et al., (Yang, 2011) developed heparin-based metallic nanoparticles having a low molecular weight protamine on the surface as cell-penetrating peptide that could be localized to the brain using a strong magnetic field (Yang, 2011). Such nanoparticles however were not shown to accumulate in the brain in the absence of any external magnetic field.

Jeon et al., (Jeon, 2006) developed heparin conjugated poly(L-lactide-co-glycolide) (PLGA) nanospheres that were used for the delivery of bFGF. Such nanoparticles however were not shown to target the CNS.

She et al., (She, 2013) have developed dendronized micelles of heparin by conjugating lysine units followed by 4-(N-tert-butoxycarbonyl-hydrazino)-4-oxo-butyric acid, which was subsequently conjugated with DOX to form a pH responsive hydrazone linkage (She, 2013). The DOX loaded dendronized nanoparticles displayed efficient tumor suppression in female BALB/c mice bearing 4T1 breast cancer models. The unloaded nanoparticles did not show any toxicity or tumor suppression.

Heparin coating of nanoparticles provides stealth properties (i.e. “invisibility” to biological systems involved in clearance of particles from the blood stream) and enhances the bioavailability as heparin coating lowers the uptake by the reticuloendothelial system cells in the liver and spleen and prevents the adsorption of serum proteins (Han, 2006). For example, heparin stabilized liposomes displayed 1.5 folds higher stability than pegylated liposomes and displayed higher accumulation in tumor tissue in murine melanoma model (B16F10) grafted in female C57BL/6 mice (Han, 2006). Chung et al., (Chung, 2010) coated PLGA nanoparticles with heparin that displayed 2.2 fold higher accumulation into SCC7 tumor-bearing athymic mice than PEGylated PLGA particles, though accumulation of both particles in liver was similar. However, biodistribution of heparin coated particles into the brain is not reported.

There still is a need for new compositions capable of crossing the BBB in a reliable and controllable way, e.g. for use in the treatment and diagnosis of CNS disorders. There also remains a critical need for minimally invasive biomarkers for diagnosis of CNS disorders and as a means for assessing response to therapeutic interventions. Better prognostic markers would allow physicians to diagnose and begin treatment of GBM at early onset, possibly preventing disease progression (Shergalis, 2018)).

### SUMMARY OF THE INVENTION

Provided herein is a nanoparticle capable of crossing the BBB, for use in therapy and diagnosis of conditions and disorders affecting the central nervous system, in particular the brain.

According to a first embodiment, a nanoparticle is provided, comprising chemically modified heparin, wherein the heparin has been chemically modified by attaching hydrophobic moieties to functional groups of the heparin, in particular selected from hydroxy groups and carboxy groups, for use in the treatment or diagnosis of a brain disorder.

According to a further embodiment, a nanoparticle is provided, comprising a core particle of metal or metal oxide having a coating of heparin, for use in the treatment or diagnosis of a brain disorder.

According to a still further aspect, the use of a nanoparticle as defined herein is provided, in the manufacture of a medicament for the treatment of a brain disorder.

According to a still further aspect, the use of a nanoparticle as defined herein is provided, in the manufacture of a diagnostic agent for the diagnosis of a brain disorder.

According to a still further aspect, a pharmaceutical composition is provided comprising a nanoparticle as defined herein for use in the treatment of a brain disorder.

According to a still further aspect, a pharmaceutical composition (e.g. a diagnostic agent) is provided comprising a nanoparticle as defined herein for use in the diagnosis of a brain disorder.

According to a still further aspect, a method for the treatment of a brain disorder is provided, comprising administering a nanoparticle as defined herein to a mammal suffering from such a disorder.

According to a still further aspect, a method for the diagnosis of a brain disorder is provided, comprising administering a nanoparticle as defined herein to a mammal in need of such diagnosis, e.g. a mammal presenting one or more symptoms of a brain disorder.

### BRIEF DESCRIPTION OF THE DRAWINGS

FIG. 1 represents a synthetic scheme of the preparation of a doxorubicin loaded heparin nanoparticle (HP—NP).

FIG. 2 is a histogram of HP-NPs using Malvern's dynamic light scattering measurements displaying hydrodynamic size (in nanometers, nm) distribution of nanoparticles prepared in Example 1.

FIG. 3 represents a synthetic strategy for the preparation of the heparin coated super paramagnetic iron oxide nanoparticles (SPIONs) of Example 2.

FIG. 4A is a graph displaying hydrodynamic size (in nanometers, nm) distribution using Malvern's dynamic light scattering measurements of SPIONs used to prepare the heparin-coated SPIONs of Example 2.

FIG. 4B is a graph displaying hydrodynamic size (in nanometers, nm) distribution using Malvern's dynamic light scattering measurements of the heparin-coated SPIONs of Example 2.

FIG. 4C is a graph displaying zeta potential of SPIONs used to prepare the heparin-coated SPIONs of Example 2.

FIG. 4D is a graph displaying zeta potential of SPIONs of the heparin-coated SPIONs of Example 2.

FIG. 5 is a schematic representation of the process for synthesizing heparin coated gold nanoparticles complexed with gadolinium ions as described in Example 4.

FIG. 6A is a graph showing hydrodynamic size distribution (in nanometers, nm) of the heparin coated gold nanoparticles prepared in Example 4, obtained using Malvern's dynamic light scattering measurements. FIG. 6B is a graph showing hydrodynamic size distribution (in nanometers, nm) of heparin coated gold nanoparticles complexed with gadolinium, obtained using Malvern's dynamic light scattering measurements. FIG. 6C is a graph showing zeta potential measured for the heparin coated gold nanoparticles prepared in Example 4. FIG. 6D is a graph showing zeta potential data of the heparin coated gold nanoparticles complexed with gadolinium prepared in Example 4.

FIGS. 7A and 7B show relative relaxivity of the heparin coated gold nanoparticles complexed with gadolinium prepared in Example 4.

FIG. 8 represents transmission electron microscopy of the heparin coated gold nanoparticles complexed with gadolinium prepared in Example 4. Scale bar 200 nm.

FIG. 9 is a schematic representation of the process for preparation of aminooxy modified heparin coated gold nanoparticles and conjugation thereof to aldehyde-modified siRNA using oxime chemistry (Example 5).

FIG. 10A is a graph showing the hydrodynamic size of the aminooxy modified heparin gold nanoparticles prepared in Example 5 (HP—Au-NPs-Aminooxy), as determined by DLS.

FIG. 10B is a graph showing the zeta potential of aminooxy modified heparin gold nanoparticles prepared in Example 5 (HP—Au-NPs-Aminooxy).

FIG. 11 is an electrophoresis image of siRNA and the siRNA-conjugated heparin coated gold nanoparticles prepared in Example 5, respectively, in a 15% native PAGE.

FIG. 12 is a schematic representation of the process for preparing ¹⁸F tagged heparin coated gold nanopartices as described in Example 6.

FIG. 13 is a bar chart showing the anti-coagulation activity, expressed as anti-FXa activity in IU/mg, of unfractionated heparin (UFH) and of the heparin nanoparticles (HP-NPs) of Example 1, respectively in non-anticoagulated human whole blood.

FIG. 14 indicates T1 and T2 relaxation obtained by MRI of mouse brain in an orthotopic glioblastoma model (A) shows T2 relaxation after 30 minutes of exposure; (B) shows brain before treating the animals with heparin coated gold nanoparticles complexed with gadolinium; and (C) shows T1 relaxation after 3 h after treating the animals with heparin coated gold nanoparticles complexed with gadolinium.

FIG. 15A is a plot of the permeability, in cm/s, of hyaluronic acid nanoparticles, (HA-NPs), chondrotin-sulfate nanoparticles (CS), heparin nanoparticles (HP) and doxorubicin-loaded heparin nanoparticles (HP-Dox), measured in an in vitro blood-brain barrier assay with a BBB model membrane comprising an endothelial cell (EC) layer on the apical side (blood side) and astrocyte layer on the basolatral side (brain side) of a Transwell® membrane insert. Sodium fluorescein (Na-Fl) was used as a reference. FIG. 15B shows confocal microscopy images of the in-vitro BBB model membrane in the presence of heparin nanoparticles (HP-NPs) and chondrotin-sulfate nanoparticles (CS-NPs), respectively.

FIGS. 16A-16D show confocal microscopy images of brain section exhibiting brain distribution of fluorescent nanoparticles in healthy FVB mice after tail vein injection of either heparin nanoparticles or chondrotin-sulfate nanoparticles. FIG. 16A shows fluorescent HP-NPs in brain 1 h after injection; FIG. 16B shows fluorescent HP-NPs in brain 24 h after injection; FIG. 16C shows fluorescent CS-NPs 1 h after injection and FIG. 16D shows fluorescent CS-NPs 24 h after injection.

FIGS. 17A-17D show the bioluminesence image of luciferase expressing patient derived BT-12 cells in the mouse brain and total brain scan using 9 μm-thick coronal sections collected from the frontal to the anteroposterior part of the brain after six tail vein injections every third day with vehicle control (FIG. 17A); or at a 5 mg/kg dose of: doxorubicin (FIG. 17B); heparin nanoparticles (FIG. 17C); or doxorubicin-conjugated nanoparticles (FIG. 17D), respectively.

FIG. 18A is a bar chart showing the number of photons registered in the bioluminescence imaging of tumors, 24 days after tumor engraftment in six-weeks-old immunocompromised NMRI-nu (Rj:NMRI-Foxn1nu/Foxn1nu, Janvier Labs) mice bearing patient derived BT12 tumor cells expressing the luciferase reporter protein, and having received treatment with PBS (Vehicle), anti-tumor agent doxorubicin (Doxorubicin), plain heparin nanoparticles (HP-NPs) or doxorubicin-modified heparin nanoparticles (HP-Dox-NPs). FIG. 18B is a bar chart showing the measured volume (in μm³) of tumors in immunocompromised nude mice having received human brain tumor cells BT12, 24 days after tumor engraftment, and having received treatment with PBS (Vehicle), anti-tumor agent doxorubicin (Doxorubicin), plain heparin nanoparticles (HP-NPs) or doxorubicin-modified heparin nanoparticles (HP-Dox-NPs).

FIGS. 19A-19D show Western blot analysis of heparin binding EGF-like growth factor (HB-EGF) expression in brain tissue of immunocompromised nude mice implanted with BT-12 cells after treatment with HP-NPs (prepared in Example 1) as described in the in-vivo glioma study herein below. FIG. 19A shows Western blot analysis HB-EGF expression in whole mouse brain using beta-tubulin as protein loading control. FIG. 19B shows identification of HB-EGF band in Western blot analysis. FIG. 19C Western blot analysis HB-EGF expression in glioma cells present in the mouse brain after treatment using human vimentin as the protein loading control. In the left-hand box in each one of FIGS. 19A to C the HB-EGF expression in BT12 cells after in-vitro culture is shown; in the mid-box, the (HB-EGF expression in healthy mouse brain is shown and in the right-hand box, the HB-EGF expression in test groups is shown, namely, vehicle control, HP-NPs (Example 1) treated group and HP-DOX-NPs (Example 1) treated groups. FIG. 19D and FIG. 19E are bar charts showing the quantification of HB-EGF expression in whole brain tissue and in human cells in the mouse brain.

FIG. 20A shows flow cytometry histogram showing the uptake of fluorescent HP-NPs (Example 1) in mouse brain endothelial cells (bEND3). FIG. 20B shows flow cytometry histogram showing the uptake of fluorescent HP-NPs (Example 1) in human glioblastoma cell line U87-MG in presence or absence of free unfractionated heparin.

FIG. 21 shows the ⁶⁸Ga radioactivity in rat plasma after tail vein injection of ⁶⁸Ga labelled HP-NPs (Example 7) in 1 h.

FIG. 22 shows decay corrected radioactivity of ⁶⁸Ga in full blood, plasma and RBCs after tail vein injection of ⁶⁸Ga labelled HP-NPs (Example 7) in 1 h.

FIG. 23 is a PET 3D projection (Maximum Intensity Projection) that shows the kinetic PET images of rat from 1 min to 90 min after tail vein injection of ⁶⁸Ga labelled HP-NPs (Example 7). Brain (Br), heart (H), liver (L) and bladder (BI) are marked in the image.

FIGS. 24A-24C show quantified biodistribution of ⁶⁸Ga labelled HP-NPs after tail vein injection in healthy rats. FIG. 24A shows the percentage of injected dose per gram tissue (% ID/g) accumulated in different organs. FIG. 24B shows percentage of injected dose (% ID) accumulated in different organs. FIG. 24C shows percentage of injected dose (% ID) accumulated in brain (filled triangles) and spleen (empty circles).

### DETAILED DESCRIPTION OF THE INVENTION

Herein below a number of publications are referred to and the content of each such publication is incorporated herein in its entirety by reference.

As used herein, the singular forms “a,” “an,” and “the” include plural reference unless the context clearly dictates otherwise, or unless otherwise specified.

“Treatment” or “treating” as used herein includes achieving a therapeutic benefit and/or a prophylactic benefit. By therapeutic benefit is meant eradication or amelioration of the underlying disorder or condition being treated. Also, a therapeutic benefit is achieved with the eradication or amelioration of one or more of the physiological or psychological symptoms associated with the underlying condition such that an improvement is observed in the patient, notwithstanding the fact that the patient may still be affected by the condition. A prophylactic benefit of treatment includes prevention of a condition, retarding the progress of a condition, or decreasing the likelihood of occurrence of a condition.

As used herein, the expression “therapeutically effective amount” refers to an amount of a nanoparticle of the invention that is sufficient to treat, ameliorate, or prevent the identified disease or condition, or that is sufficient for a detectable therapeutic, prophylactic, or inhibitory effect to be exhibited. The effect can be detected by, for example, an improvement in clinical condition, or reduction in symptoms. The precise effective amount for any treated subject will depend upon factors such as the body weight, size, age, and general condition of the subject; as well as the nature and extent of the condition to be treated and will generally be determined by the treating physician.

As used herein, the reference to a patient “in need of a nanoparticle that is able to cross the blood-brain barrier” relates to a patient who would benefit from treatment with such a nanoparticle. The patient may be suffering from any disease or condition for which therapy with a nanoparticle that is able to cross the blood-brain bather may be useful in ameliorating symptoms.

As used herein, the term “blood-brain barrier” (BBB) refers to the barrier between the peripheral circulation and the brain and spinal cord which is formed by tight junctions within the brain capillary endothelial plasma membranes, which create a highly selective barrier that restricts the transport of molecules into the brain. The blood-brain barrier within the brain, the blood-spinal cord barrier within the spinal cord, and the blood-retinal barrier within the retina, are contiguous capillary barriers within the central nervous system (CNS), and may collectively be referred to as the blood-brain barrier or BBB.

As used herein, the expression “brain disorder” (or e.g. disorder affecting the brain, or similar expression) refers to any disorder or disease located in the brain, e.g. a malignant hyperproliferative disorder, in particular a malignant glioma, such as a glioblastoma, e.g. GBM; a cognitive disorder, such as a dementia; or a neuroinflammatory disorder. For example, the brain disorder may be Alzheimer's disease.

As used herein, the term “mammal” includes humans and non-human mammals, such as primates, domesticated animals such as farm animals, e.g. cattle, sheep, pigs, horses and the like, as well as pet animals, such as dogs and cats, and the like. Preferably, the mammal is a human.

As used herein, the term “hydroxy group” refers to the functional group of formula:

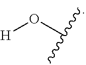 [SMILES: *O]

As used herein, the term “carboxy group” refers to the functional group of formula:

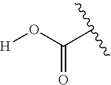 [SMILES: *C(=O)O]

The unit kDa, as used herein, corresponds to 10³ daltons. A dalton (Da) is a unit of mass equal to one twelfth of the mass of an atom of ¹²C. It is equivalent to approximately 1.66×10⁻²⁷ kg.

A first aspect relates to a nanoparticle that comprises chemically modified heparin, wherein the heparin has been chemically modified by attaching hydrophobic moieties to functional groups of the heparin, said functional groups being selected from in particular hydroxy groups and carboxy groups, for use in the treatment or diagnosis of a brain disorder.

Heparin, also referred to as unfractionated heparin, is a naturally occurring linear highly sulphated polysaccharide, a glycosaminoglycan consisting of a heterogenous chain of (1-4)-linked disaccharide repeating units of uronic acid (either d-glucuronic acid or 1-iduronic acid) and d-glucosamine residues. It is produced endogenously by basophils and mast cells and has been extensively used in pharmacy, in particular as an anticoagulant. Native heparin has a molecular weight ranging from about 3 to 30 kDa. The most common disaccharide unit is composed of a 2-O-sulfated iduronic acid and 6-O-sulfated, N-sulfated glucosamine, IdoA(2S)-GIcNS(6S), though other types of disaccharides may also be present.

Heparin as used in the present invention has a molar mass (“molecular weight”) of at least 8 kDa, more preferably at least 10 kDa, at least 12 kDA, or at least 15 kDA, e.g. from 8 to 30 kDa, or from 10 to 30 kDa, or from 12 to 30 kDa, or from 15 to 30 kDA. In some embodiments, the heparin for use according to the invention has a molar mass of at most 25 kDa, or at most 20 kDa, or at most 18 kDa.

The number of disaccharide units in heparin as used herein normally ranges from more than 10 to about 50. In some embodiments, the number of disaccharide units in a heparin molecule is at least 15, at least 20, at least 25, or at least 30. In some embodiments, the number of disaccharide units in a heparin molecule is at most 45, or at most 35, at most 30, or at most 25. In some embodiments, thus, a heparin molecule as used herein contains from about 15 to about 50 disaccharide units; or from about 20 to about 50, from about 15 to about 40, from about 20 to about 40; from about 15 to about 30, or from about 20 to about 30 disaccharide units.

It should be realized that the heparin polymer contains numerous carboxy and hydroxy groups distributed along the polymeric backbone, and these groups may be modified by, for example, etherification, esterification, acylation or amidation. By selection of suitable reaction components and reaction conditions, hydrophobic moieties may be attached to functional groups of the heparin, to provide a heparin molecule substituted with hydrophobic groups.

In some embodiments, thus, heparin molecules as used herein are modified essentially by attachment of hydrophobic groups to hydroxy groups of the heparin polysaccharide, e.g. by etherification or acylation of said hydroxy groups.

In some particular embodiments, the modified heparin molecule essentially comprises hydroxy groups modified by attachment of a hydrophobic group by acylation.

In some further embodiments, heparin molecules as used herein are modified essentially by attachment of hydrophobic groups to carboxy groups of the heparin polysaccharide, e.g. by esterification or amidation, of said carboxy groups (i.e. transformation of carboxy groups (i.e. carboxylic acid functions, C(O)OH) to carboxylic ester functions (C(O)OR₁) or carboxamide functions (CONR₂R₃), where R₁ and at least one of R₂ and R₃ is a hydrophobic moiety as defined herein.

In some of these embodiments, R₁ and at least one of R₂ and R₃ are selected from C8 to C28 aliphatic groups, e.g. C8 to C20 aliphatic groups, or C8 to C18 aliphatic groups. For example, any such aliphatic groups may be selected from linear, branched, partly cyclic or cyclic (e.g. monocyclic or polycyclic), saturated or unsaturated (e.g. mono-, di- or tri-unsaturated) aliphatic group; while one of R₂ and R₃ may be H or e.g. C1-C3 alkyl.

Reactions for transforming carboxy groups to, for example, esters or amides, are well known to the person of ordinary skill, and e.g. may be performed by use of suitable carbodiimde derivatives. Other derivatization reactions are also considered possible, e.g. reaction with a hydrazinecarbothioamide derivative of formula R—NHC(S)NH(NH₂), wherein R is a hydrophobic moiety or e.g. a fluorescent moiety.

In some embodiments, the modified heparin molecules may have both carboxy groups and hydroxy groups that are modified as described herein.

The chemical modification of the heparin molecules as described herein is such as to allow—or lead to—formation of micellar nanoparticles, by auto-assembly of the modified heparin. Heparin-based nanoparticles and methods for their preparation have been previously described, cf. e.g. (Kemp, 2010).

The degree of substitution with hydrophobic groups of the heparin generally ranges from about 1% to about 10%, based on the number of hydroxy groups and carboxy groups of the corresponding unsubstituted heparin. In some embodiments, the degree of substitution is at least 2%, at least 3%, at least 4%, at least 5%, at least 6%, at least 7%, or even at least 8%, based on the number of hydroxy groups and carboxy groups of the corresponding unsubstituted heparin. In some embodiments, the degree of substitution of the heparin with hydrophobic groups is at most 9%, at most 8%, at most 7%, at most 6% at most 5%, or at most 4%, based on the number of hydroxy groups and carboxy groups of the corresponding unsubstituted heparin.

It should be noted that the substituents may be not uniformly distributed over the entire heparin molecule. However, in some embodiments, at least 30% of the disaccharide units of any heparin molecule are substituted by hydrophobic groups, e.g. at least 25%, at least 20%, at least 10%, at least 5%, or at least 2% of the disaccharide units the heparin molecule are substituted by one or more hydrophobic groups. Thus, in some embodiments, the degree of substitution of the heparin molecule ranges from about 1% to about 30%, e.g. about 1% to about 25%, or about 1% to about 20%, e.g. about 1% to about 10%, where the degree of substitution refers to the percentage of disaccharide units of the heparin molecule carrying at least one hydrophobic substituent.

It should be realized that within the above indicated ranges, the heparin molecules making up any particular nanoparticle may vary in degree of substitution and distribution of hydrophobic substituents over the polysaccharide chain, as well as specific type of hydrophobic substituents, e.g. in case the heparin molecules are substituted by more than one type of hydrophobic substituent. The degree of substitution may be measured e.g. by nuclear magnetic resonance (NMR) or by elemental analysis.

In some embodiments, the hydrophobic groups are derived from lipids, such as cholesterol, ceramides, and fatty acids; fluorescein and fluorescein derivatives; and hydrophobic polymers.

In some embodiments, the hydrophobic groups attached to heparin derived from lipids, e.g. a sterol, such as cholesterol, a fatty acid, such as a C8 to C28 fatty acid, or a ceramide, such as a ceramide of a C8 to C28 fatty acid.

In some embodiments, the hydrophobic groups attached to heparin may comprise a sterol, in particular cholesterol. A method for preparing a cholesterol-modified polysaccharide is described in (Shaikh, 1998), which method may also be applied to prepare suitably cholesterol-modified heparin molecules for use as described herein.

In some embodiments, the hydrophobic groups attached to heparin may comprise fluorescein, or a fluorescein derivative, such as fluorescein-5-thiosemicarbazide or fluorescein isothiocyanate. For example, a method for modifying a polysaccharide by attachment of fluorescein is described in (Ding, 2016).

In some embodiments, the hydrophobic groups comprise groups of the type R—C(O)— derived from one or more fatty acids. The fatty acid R—C(O)OH (corresponding to the fatty acid residue R—C(O)—) may be of any origin, e.g. derived from a vegetable oil, and may be e.g. saturated, mono-unsaturated, di-unsaturated, or tri-unsaturated. In some embodiments, the fatty acid is unsaturated, e.g. mono-unsaturated, di-unsaturated, or tri-unsaturated. In some embodiments, the fatty acid is di-unsaturated. In some other embodiments, the fatty acid is mono-unsaturated. The fatty acid preferably has an unbranched (linear) chain and contains from e.g. 8 to 26 carbon atoms. In some embodiments, the number of carbon atoms in the fatty acid is at least 10, at least 12, at least 14, or at least 16. In some embodiments, the number of carbon atoms in the fatty acid is at most 24, at most 22, at most 20 or at most 18.

In some embodiments, any saturated fatty acid R—C(O)OH (the residue of which is) contemplated for use according to the invention is selected from fatty acids having from 8 to 24 carbon atoms, or from 8 to 22 carbon atoms, or from 8 to 20 carbon atoms, or from 8 to 18 carbon atoms, or from 8 to 16 carbon atoms or from 8 to 14 carbon atoms. In some embodiments, a saturated acid is selected from fatty acids having from 10 to 24 carbon atoms, or from 10 to 22 carbon atoms, or from 10 to 20 carbon atoms, or from 10 to 18 carbon atoms, or from 10 to 16 carbon atoms or from 10 to 14 carbon atoms. In some embodiments, a saturated acid R—C(O)OH is selected from fatty acids having from 12 to 24 carbon atoms, or from 12 to 22 carbon atoms, or from 12 to 20 carbon atoms, or from 12 to 18 carbon atoms, or from 12 to 16 carbon atoms. In some embodiments, a saturated acid R—C(O)OH is selected from fatty acids having from 14 to 24 carbon atoms, or from 14 to 22 carbon atoms, or from 14 to 20 carbon atoms, or from 14 to 18 carbon atoms.

Examples of saturated fatty acids contemplated for use according to the invention are caprylic acid, capric acid, lauric acid, myristic acid, palmitic acid, stearic acid, arachidic acid, behenic acid and lignoceric acid.

In some embodiments, the fatty acids also may be selected from unsaturated fatty, acids. In some embodiments, an unsaturated fatty acid R—C(O)OH has a number of carbon atoms ranging from 10 to 24, e.g. from 10 to 22, or from 10 to 20, or from 10 to 18. In some embodiments, the unsaturated fatty acid has a number of carbon atoms ranging from 12 to 24, e.g. from 12 to 22, or from 12 to 20, or from 12 to 18. In some embodiments, an unsaturated fatty acid has a number of carbon atoms ranging from 14 to 24, e.g. from 14 to 22, or from 14 to 20, or from 14 to 18. In some embodiments, an unsaturated fatty acid has from 16 to 24 carbon atoms, or from 16 to 22 carbon atoms, e.g. from 16 to 20 carbon atoms, e.g. 16 or 18 carbon atoms.

In some embodiments, an unsaturated fatty acid has a number of carbon atoms as indicated herein above and 1-5 carbon-carbon double bonds, e.g. 1-4 carbon-carbon double bonds, or 1-3 carbon-carbon double bonds, e.g. 1 or 2 carbon-carbon double bonds. In some embodiments, an unsaturated fatty acid is mono-unsaturated (i.e. it has one (1) carbon-carbon double bond). In some embodiments, an unsaturated fatty acid is diunsaturated (i.e. it has two (2) carbon-carbon double bonds). In some embodiments, a fatty acid is a C18 fatty acid, e.g. a C18 mono- or diunsaturated fatty acid, in particular a C18 monounsaturated fatty acid. In some particular embodiments, a fatty acid is a C14-C24 mono- or diunsaturated fatty acid, e.g. a C14-C22 mono- or diunsaturated fatty acid, or a C16-C22 mono- or diunsaturated fatty acid, e.g. a C16-C20 mono- or diunsaturated fatty acid.

Examples of unsaturated fatty acids contemplated for use according to the invention are myristoleic acid, palmitoleic acid, oleic acid, elaidic acid, vaccenic acid, linoleic acid, linoelaidic acid, α-linolenic acid, arachidonic acid, eicosapentaenoic acid, erucic acid, and docosahexaenoic acid.

In some embodiments, the hydrophobic groups attached to heparin may comprise a ceramide, i.e. sphingosine, the amino group of which has been acylated by a fatty acid, e.g. a fatty acid selected from the fatty acids described herein above.

In some embodiments, the hydrophobic groups attached to heparin may comprise a hydrophobic polymer, e.g. selected from poloxamer, e.g. (“pluronics”), poly-β-benzyl-1-aspartate, poly-γ-benzyl-1-glutamate, and poly(lactic-co-glycolic acid).

In some embodiments, the hydrophobic polymer is or comprises a poloxamer. Poloxamers are nonionic triblock copolymers composed of a central hydrophobic chain of polyoxypropylene flanked by two hydrophilic chains of polyoxyethylene. The polymer may be represented by the formula HO—[CH₂CH₂O]ₐ—[CH(CH₃)CH₂O]_{y}—[CH₂CH₂O]ₓ—H, where x generally is an integer of from 2 to 130 and y generally is an integer of from 15-67. Poloxamer-conjugated heparin and a method for its preparation is described in (Choi, 2011). Poloxamers are commercially available, e.g. as Pluronic® F-127 CAS Number: 9003-11-6.

In some embodiments, the hydrophobic polymer is selected from poly-β-benzyl-1-aspartate and poly-γ-benzyl-1-glutamate or is a copolymer of poly-β-benzyl-1-aspartate and poly-γ-benzyl-1-glutamate. The synthesis and identification of such poly-amino acids are described in (Wang, 2008).

In some embodiments, the hydrophobic polymer is a poly(lactic-co-glycolic acid) (PLGA). PLGA may be synthesized as a random or block copolymer by means of ring-opening co-polymerization of the cyclic dimers (1,4-dioxane-2,5-diones) of glycolic acid and lactic acid. The synthesis and characterization of PLGA, as well as the conjugation of PLGA to heparin are described in (Jeon, 2006).

In addition to the hydrophobic groups attached to the heparin, further moieties, may also be attached, e.g. labelling groups for use in a diagnostic method, as mentioned herein.

The nanoparticle of the invention may be physically or chemically attached to of one or more biologically or therapeutically active compounds, e.g. an anticancer agent or a compound for use in the treatment of cognitive disorders. For example, the nanoparticle may be physically or chemically attached to a compound selected from small molecule drugs such as doxorubicin or cisplatin, or large molecule drugs such as nucleic acids (e.g. siRNA), antibodies, peptides or proteins, or to a diagnostic agent.

In some embodiments, thus, the nanoparticle of the invention is used as vehicle or carrier for one or more therapeutically active agents and/or diagnostic agents, allowing for an efficient delivery thereof into the CNS system.

In some embodiments, the therapeutically active agent is an anticancer agent, such as doxorubicin, cisplatin, or temozolomid.

In some embodiments, the therapeutically active agent is an anticancer agent, useful for the treatment of a brain tumor, such as GBM. However, it should be realized that the heparin nanoparticle of the invention per se, i.e. without carrying any other therapeutically active agent, has a surprising anti-tumor effect, e.g. for the treatment of a brain tumor such as GBM.

In some embodiments, the therapeutically active agent is an agent that is useful for the treatment of a cognitive disorder, such as dementia, in particular Alzheimer's disease.

In some embodiments, the therapeutically active agent is a large molecule drug.

Chemical attachment may be by e.g. ionic binding, hydrogen binding or, covalent binding. For example, covalent binding may be achieved by attachment to any of the functional groups of the heparin molecule, e.g. the hydroxy groups, or the carboxy groups in the same way as the attachment of the hydrophobic groups.

Physical attachment may include e.g. electrostatic binding, or by (micellar) inclusion within the heparin nanoparticle.

A nanoparticle according to the present invention may be a micellar nanoparticle formed by assembly of chemically modified heparin as defined herein or may comprise a metal or metal oxide core having a heparin coating.

In some embodiments, the nanoparticle is a micellar nanoparticle formed by assembly of chemically modified heparin as defined herein.

In some other embodiments, the nanoparticle is a metal or metal oxide core having a heparin coating, e.g. a gold nanoparticle, gadolinium oxide nanoparticle, or an iron oxide particle having a heparin surface coating, optionally modified by attachment of one or more chemical moieties to the heparin. It should be realized that in such embodiments, the heparin (which is as defined herein) may be unmodified, i.e. carrying no hydrophobic groups of the type disclosed herein. Thus, in some embodiments, the nanoparticle is a metal or metal oxide core having a coating of heparin that has not been modified by attachment of hydrophobic groups.

In some embodiments, the heparin may be attached to the metal or metal oxide core by modifying the heparin molecule with groups capable of binding to the metal or metal oxide, e.g. thiol-containing groups capable of chelating gold or gadolinium. Thus, for example, a metal or metal oxide nanoparticle may be coated with heparin that has been modified by attaching 3,3′-dithiobis(propanoic hydrazide), e.g. using carbodiimide chemistry.

In some embodiments, the metal core is a gold nanoparticle. A review of the applications of gold nanoparticles as a tool in cancer diagnostics and treatment is provided in (Singh, 2018). Coating of gold nanoparticles with heparin has been described e.g. in (Kemp, 2009).

Heparin coated gold nanoparticles may also be tagged with a radioactive isotope, such as the radioactive fluorine isotope ¹⁸F for PET imaging. Thus, in some embodiments, nanoparticles are provided, comprising a gold core coated with heparin, wherein the heparin is modified by attachment of a ¹⁸F-containing group, e.g. ¹⁸F-fluorodeoxyribose. Such particles may allow for efficient detection and localization of a brain tumor.

To attach ¹⁸F-fluorodeoxyribose to heparin, the heparin molecule may first be modified by attachment of 4-(2-aminoethyl)benzene-1,2-diol (dopamine) to the heparin carboxylate groups by carbodiimide chemistry and ¹⁸F-fluorodeoxyribose may then be attached to the dopamine aminooxy linker by oxime chemistry.

In some embodiments, the metal oxide core is a superparamagnetic iron oxide nanoparticle, e.g. magnetite (Fe₃O₄) or maghemite (γ-Fe₂O₃) nanoparticle. The coating of magnetic iron oxide particles with heparin and the physicochemical characterization of the obtained coated particles are described in (Min, 2010).

In some embodiments, the metal oxide core is a gadolinium oxide nanoparticle, such as described e.g. in (Gayathri 2015).

In some other embodiments, the nanoparticle of the invention may comprise a metal or metal oxide core, said core being coated by heparin, wherein the heparin is modified by attachment of a chemical moiety, e.g. a labelling group, such as a gadolinium label, or a radioactive moiety.

In general, the size of the nanoparticle (heparin micellar particle or metal or metal oxide core having a heparin coating) is in the range of from 40 to 200 nm, e.g. from 50 to 200 nm. In some embodiments, the nanoparticle has a size of at least 60 nm. In some embodiments, the nanoparticle has a size of at least 70 nm. In some embodiments, the nanoparticle has a size of at least 80 nm. In some embodiments, the nanoparticle has a size of at least 90 nm. In some embodiments, the nanoparticle has a size of at least 100 nm. In some embodiments, the nanoparticle has a size of at least 110 nm. In some embodiments, the nanoparticle has a size of at least 120 nm. In some embodiments, the nanoparticle has a size of at least 130 nm.

In some embodiments, the nanoparticle has a size of at most 190 nm, at most 180 nm, at most 170 nm, at most 160 nm, or at most 150 nm. Thus, the nanoparticle may have a size ranging between any of the indicated lower and upper limits. For example, in some embodiments, the nanoparticle has a size within the range of from 70 nm to 180 nm, or from 80 nm to 170 nm; or from 90 nm to 160 nm, or from 100 nm to 150 nm, or from 110 nm to 150 nm, or from 120 nm to 150 nm, or from 130 nm to 150 nm, e.g. about 140 nm.

The heparin nanoparticles of the invention have a surprisingly high capacity of crossing the BBB, which forms the basis for the use of these nanoparticles in the treatment or diagnosis of various brain disorders.

For example, it is contemplated that the nanoparticles provided herein will be of use in the treatment and diagnosis of various brain disorders, e.g. in brain cancer magnetic nanotherapy, brain tumor targeting, and in medical imaging, as well as in magnetic hyperthermia as a brain cancer treatment method. The nanoparticles of the invention are contemplated for the treatment of brain disorders, such as malignant hyperproliferative disorders (e.g. malignant gliomas, such as glioblastoma, e.g. GBM; neuroinflammatory diseases; and cognitive diseases such as various dementias, in particular Alzheimer's disease.

Additionally, it is contemplated that the nanoparticles provided herein will be of use for delivering biologics across the BBB, such as nucleic acids, antibodies, peptides and proteins, for the treatment of brain disorders.

Therefore, a composition is provided comprising the nanoparticle of the invention and a pharmaceutically carrier, for administration to a subject in need of a nanoparticle able to cross the blood-brain barrier.

The present invention includes pharmaceutical compositions comprising a therapeutically effective amount of nanoparticles of the invention, optionally together with a pharmaceutically acceptable excipient, e.g. a carrier, and optionally other therapeutic and/or prophylactic ingredients, and the use thereof in the treatment of disorders affecting the brain.

A pharmaceutical composition according to the invention may be for local or systemic administration, e.g. for enteral administration, such as rectal or oral administration, or for parenteral administration to a mammal (especially a human), and comprises a therapeutically effective amount of a nanoparticle of the invention, as active ingredient, in association with a pharmaceutically acceptable excipient, e.g. a pharmaceutically acceptable carrier. The therapeutically effective amount of the active ingredient is as defined herein above and depends e.g. on the species of mammal, the body weight, the age, the individual condition, individual pharmacokinetic data, the disease to be treated and the mode of administration.

For enteral, e.g. oral, administration, the nanoparticle may be formulated in a wide variety of dosage forms. The pharmaceutically acceptable carriers may be either solid or liquid. Solid form preparations include powders, tablets, pills, lozenges, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid which is a mixture with the active ingredient. In tablets, the active ingredient generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The formulation of the active ingredient may comprise an encapsulating material as carrier, providing a capsule in which the active ingredient is surrounded by a carrier.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Liquid suspensions can be prepared by dispersing nanoparticle in a suitable liquid carrier (e.g. water), optionally with viscous material, such as natural or synthetic gums, resins, methylcellulose, sodium carboxymethylcellulose, and other well-known suspending agents. Solid form preparations include solutions, suspensions, and emulsions, and may contain, in addition to nanoparticle of the invention, colorants, flavors, stabilizers, buffers, artificial and natural sweeteners, dispersants, thickeners, solubilizing agents, and the like.

The nanoparticle of the invention also may be administered parenterally, e.g. by inhalation, injection or infusion, e.g. by intravenous, intraarterial, intraosseous, intramuscular, intracerebroventricular, intrasynovial, intrasternal, intrathecal, intralesional, intracranial, intratumoral, intracutaneous or subcutaneous injection or infusion.

Thus, for parenteral administration, the pharmaceutical compositions of the invention may be in the form of a sterile injectable or infusible preparation, for example, as a sterile aqueous or oleaginous suspension. This suspension may be formulated according to techniques known in the art using suitable dispersing or wetting agents (e.g., Tween 80), and suspending agents. The sterile injectable or infusible preparation may also be a sterile injectable or infusible solution or suspension in a non-toxic parenterally acceptable diluent or solvent. For example, the pharmaceutical composition may be a solution in 1,3-butanediol. Other examples of acceptable vehicles and solvents that may be employed in the compositions of the present invention include, but are not limited to, mannitol, water, Ringer's solution and isotonic aqueous sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant.

Liquid formulations for parenteral use also may contain suitable stabilizing agents, and if necessary, buffer substances. Suitable stabilizing agents include antioxidizing agents, such as sodium bisulfate, sodium sulfite or ascorbic acid, either alone or combined, citric acid and its salts and sodium EDTA. Parenteral solutions may also contain preservatives, such as benzalkonium chloride, methyl- or propyl-paraben, and chlorobutanol.

For inhalation or nasal administration, suitable pharmaceutical formulations are as particles, aerosols, powders, mists or droplets, e.g. with an average size of about 10 μm in diameter or less. For example, compositions for inhalation may be prepared as solutions in saline, employing benzyl alcohol or other suitable preservatives, absorption promoters to enhance bioavailability, fluorocarbons, and/or other solubilizing or dispersing agents known in the art.

Preparation of the nanoparticles of the invention may include a step of lyophilization, to provide a nanoparticle formulation in powder form, e.g. for reconstitution using a liquid phase, such as water for injection, or e.g. for inclusion in a capsule, optionally together with a suitable pharmaceutical excipient.

Suitable pharmaceutical excipients, e.g. carriers, and methods of preparing pharmaceutical dosage forms are described in Remington's Pharmaceutical Sciences, Mack Publishing Company, a standard reference text in art of drug formulation.

The pharmaceutical compositions may comprise from approximately 1% to approximately 95%, preferably from approximately 20% to approximately 90% of nanoparticles, together with a pharmaceutically acceptable excipient. In general, the nanoparticle of the invention will be administered in a therapeutically effective amount by any of the accepted modes of administration for agents that serve similar utilities. Suitable dosages may typically range from 1 to 1000 mg, e.g. 1-500 mg, or 1-50 mg, bi-daily (i.e. twice a day), daily, weekly, or less frequently, depending upon numerous factors such as the severity of the disease to be treated, the age and relative health of the patient, the route and form of administration, and the indication towards which the administration is directed, etc., and will normally be determined by the treating physician.

In some embodiments, the nanoparticles of the invention are used in the diagnosis of diseases and conditions affecting the brain, e.g. brain tumors, for example by imaging techniques such as functional magnetic resonance imaging (MRI) and diffusion tensor imaging (DTI), during preparatory phase, and ultrasound, computed tomography (CT) and MRI during surgery. Other methods where the nanoparticles of the invention may be used include positron-emission tomography (PET). In such methods, the nanoparticles may be administered to the subject e.g. by injection or infusion, as described herein above.

In some embodiments, the nanoparticle of the invention may be used in combination with radiation therapy of a malignant hyperproliferative CNS disorder, such GBM.

### EXAMPLES

The invention will be further illustrated by the following, non-limiting examples.

### Example 1

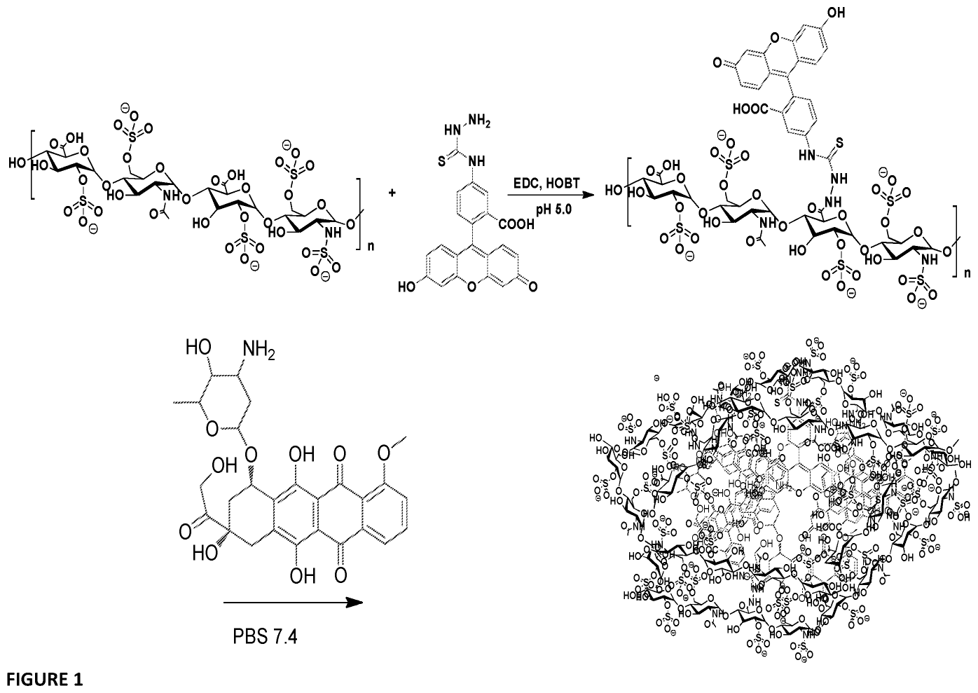

FTSC-Labelled Heparin Nanoparticles (HP-NPs) Loaded with Doxorubicin (FIG. 1)

Heparin (400 mg, 0.66 mmol) was dissolved in 150 mL of distilled water, followed by the addition of hydroxybenzotriazole (HOBT) (90 mg, 0.66 mmol) and stirred for 30 min until the reaction mixture was homogeneous. Fluorescein-5-thiosemicarbazide (FTSC) (42 mg, 0.1 mmol) was separately dissolved in 75 mL of dimethylsulfoxide (DMSO) and added to the heparin solution. The reaction mixture was stirred for 60 min and thereafter the pH of the reaction mixture was adjusted to pH 5 by dropwise addition of 1M NaOH. Finally, 1-ethyl-3-(3-dimethylaminopropyl)carbodiimide (EDC) (38.34 mg, 0.2 mmol) was added in 3 batches (20 min. interval) in order to maximize the coupling efficiency. The reaction mixture was stirred overnight, and transferred to a dialysis bag (Spectra Por-6, MWCO 3500 g/mol) and dialyzed against dilute HCl (pH=3.5) containing 100 mM NaCl (4×2 L, 24 h), followed by dialysis against diluted HCl (pH 3.5) (4×2 L, 24 h) and then dialyzed against deionized water (2×2 L, 24 h). The solution was lyophilized, and fluffy yellow material was obtained in nearly quantitative yield. This product was finally washed with methanol and filtered and dried to remove any traces of unreacted FTSC. Percentage of FTSC conjugation was estimated by UV measurement (at pH 8.5 in water) using the FTSC extinction coefficient of 78 000 M⁻¹cm⁻¹ at 492 nm. Following the same protocol, hyaluronic acid-FTSC conjugates and chondroitin sulfate-FTSC conjugates were prepared forming nanoparticles of 123 nm and 146 nm respectively as reported in ACS Appl. Mater. Interfaces 2016, 8, 20614-20624.

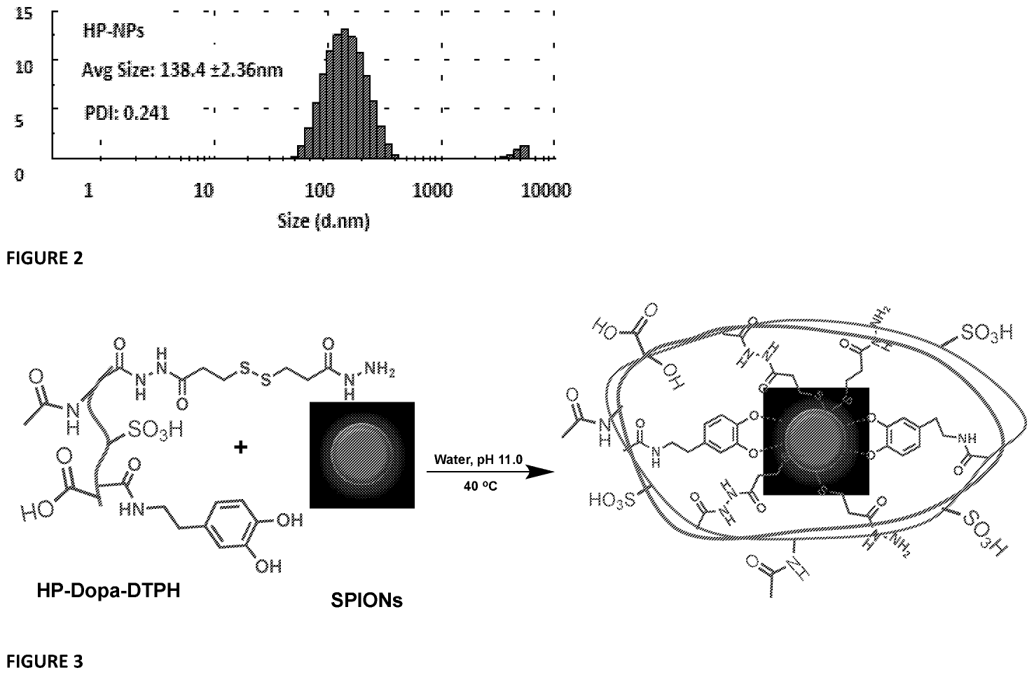

The particle size of nanoparticles was determined by dynamic light scattering (DLS) (Malvern Zetasizer Nano ZS). The freeze-dried HP-nanoparticles were dissolved in deionized water at concentration of 1 mg/ml and filtered by 0.45 μm filter before performing DLS. The average size of the HP-NPs was found to be 138.4±2.36 nm (FIG. 2).

Loading of the HP-NPs with DOX was performed by the nanoprecipitation method. Briefly, 100 mg of HP-NPs were dissolved in 25 mL of phosphate buffer saline (PBS) at pH 7.4. Thereafter, 5 mg of DOX.HCl (in 1 mL of DMSO) were added dropwise under magnetic stirring (1000 rpm). The pH was adjusted again to 7.4, and the reaction mixture was stirred overnight. Thereafter, the solution was loaded into a dialysis bag (Spectra Por-6, MWCO 3500) and dialyzed against 100 mM NaCl (2×2 L, 24 h), followed by dialysis against deionized water (2×2 L, 24 h). This solution was lyophilized yielding ˜96 mg of orange-yellow fluffy product (96% yield). The average size of the HP-Dox-NPs was found to be 117.1±0.48 nm as determined by DLS.

### Example 2

Heparin Coated SPIONs

Iron (III) chloride hexahydrate (1 mM, 271 mg) and Iron (II) chloride tetrahydrate (0.5 mM, 99 mg) were dissolved in 10 ml deoxygenated distilled water. Then 2.5 ml of 5 M NaOH solution were added dropwise to the reaction mixture under vigorous stirring at 60° C. under N₂ atmosphere. The formation of the nanoparticles (SPIONs) was visible by change in the solution color from straw yellow to dark black.

Freshly prepared SPIONs are prone to oxidation. To stabilize and prevent SPION oxidation, unfractionated heparin was coated on the nanoparticle surface. To achieve this, heparin polymer was covalently grafted with dopamine (10% with respect to the carboxylate groups of the heparin) and 3,3′-dithiobis(propanoic hydrazide) (DTPH) (8% with respect to the carboxylate groups of the heparin) using carbodiimide chemistry. The inherent adhesive property of dopamine facilitated efficient coating of SPIONs (Mazur, 2013) while the free sulfates and carboxylates provided the necessary colloidal stability.

The heparin coated colloidal solution was subsequently sonicated for 5 minutes. Thereafter, HP-SPIONs were isolated by applying an external magnetic field and the HP coated particles were washed five times with distilled water to remove the excess Fe(II), Fe(III), free polymers and other chemicals. Finally, the obtained heparin coated nanoparticles (heparin-SPION-NPs) were dried under vacuum (FIG. 3).

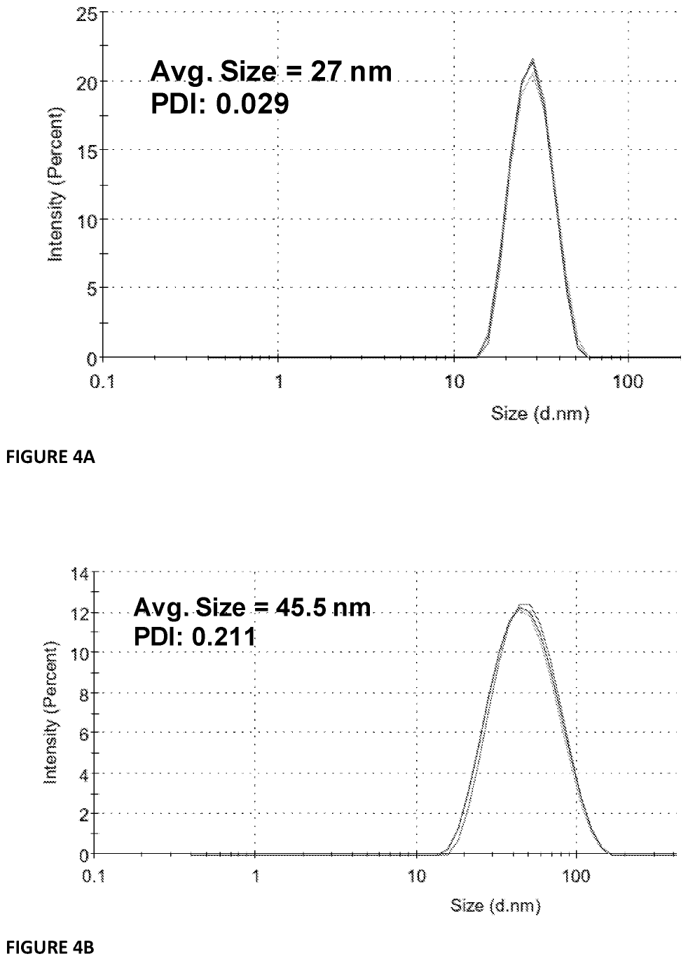

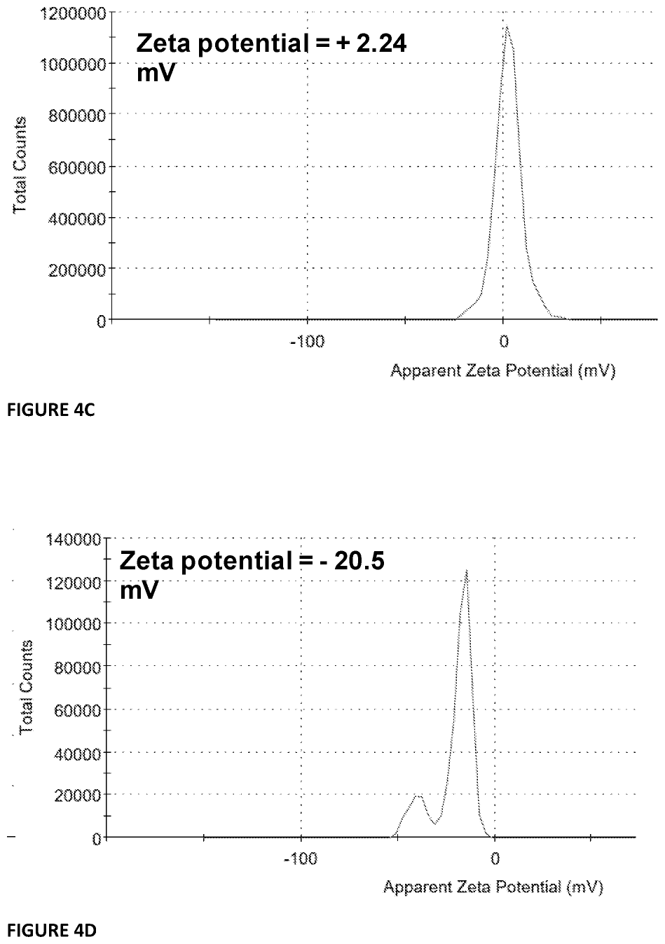

The particle size and zeta potential of SPION with and without heparin coating were determined by dynamic light scattering (DLS) (Malvern Zetasizer Nano ZS). The freeze-dried HP-nanoparticles were dissolved in deionized water at concentration of 1 mg/ml and filtered by 0.45 μm filter before performing DLS. The average size of the particles was found to be 27 nm and 45 nm for SPIONs and heparin-coated SPIONs, respectively (FIGS. 4A and B). As a result of coating, the zeta potential changed from +2.24 mV for the SPIONs to −20.5 mV for the heparin-coated SPIONs (FIGS. 4C and D).

### Example 3

Heparin Coated Gadolinium Oxide Nanoparticles

Heparin coated Gd₂O₃ nanoparticles are synthesized as follows: (1) a precursor solution of GdCl₃.6H₂O (1 mmol, 370 mg) in triethylene glycol (TEG) (10 mL) is prepared in a 50 mL three-neck-flask and (2) a solution of NaOH (3 mmol, 120 mg) in TEG (5 mL) is prepared in a beaker. Solution (1) is magnetically stirred under atmospheric conditions until complete dissolution of GdCl₃.6H₂O and subsequently treated with solution (2). The resulting mixture is heated to 80° C. for 2 h with magnetic stirring, followed by dropwise addition of 30 wt % H₂O₂ (1.8 mL) and further 2 h stirring at the same temperature. The reaction mixture is then cooled to room temperature, treated with 100 mg of heparin-Dopa-DTPH (i.e. heparin modified by attachment of 3,3′-dithiobis(propanoic hydrazide) (DTHP) and dopamine to the heparin carboxylate residues using carbodiimide chemistry), and heated to 50° C. for 24 h upon stirring, followed by cooling to room temperature and dilution with ethanol (400 mL). The produced nanoparticles are allowed to settle, and the transparent supernatant is decanted, allowing unreacted precursors, free polymers, and solvent to be effectively removed. The nanoparticle precipitate is washed with ethanol two more times, followed by washing with triply distilled water three times.

### Example 4

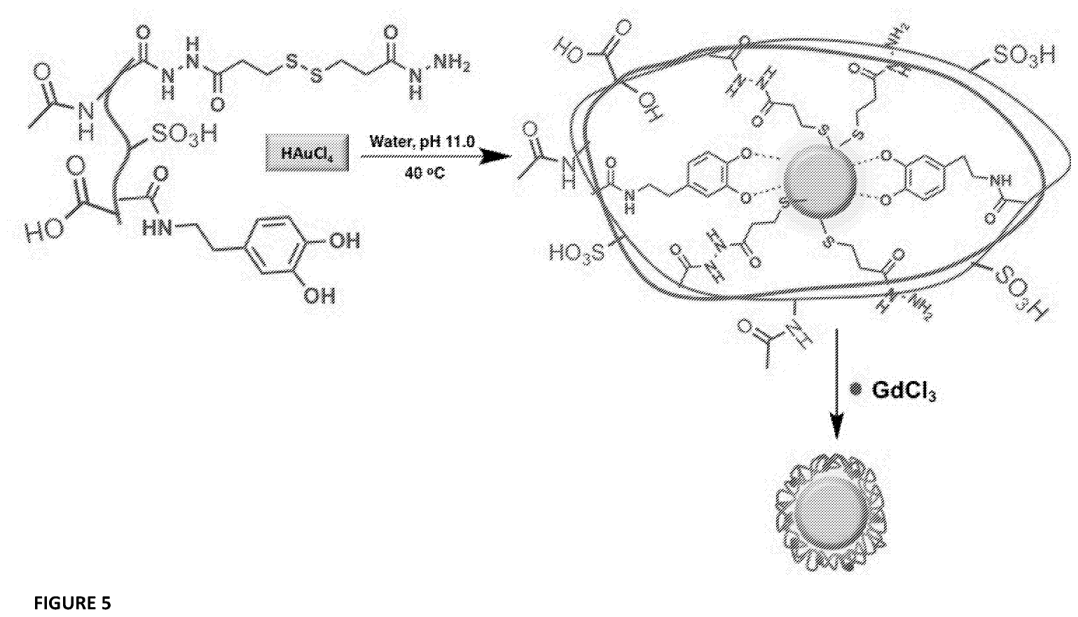

Heparin Coated Gold Nanoparticles Complexed with Gadolinium (FIG. 5)

Heparin coated gold nanoparticles were prepared using unfractionated heparin conjugated with DTPH and dopamine. The DTPH and the dopamine were conjugated to the carboxylate residues of heparin (HP) (8% and 11% respectively with respect to the carboxylate groups) following carbodiimide chemistry to obtain HP-Dopa-DTPH.

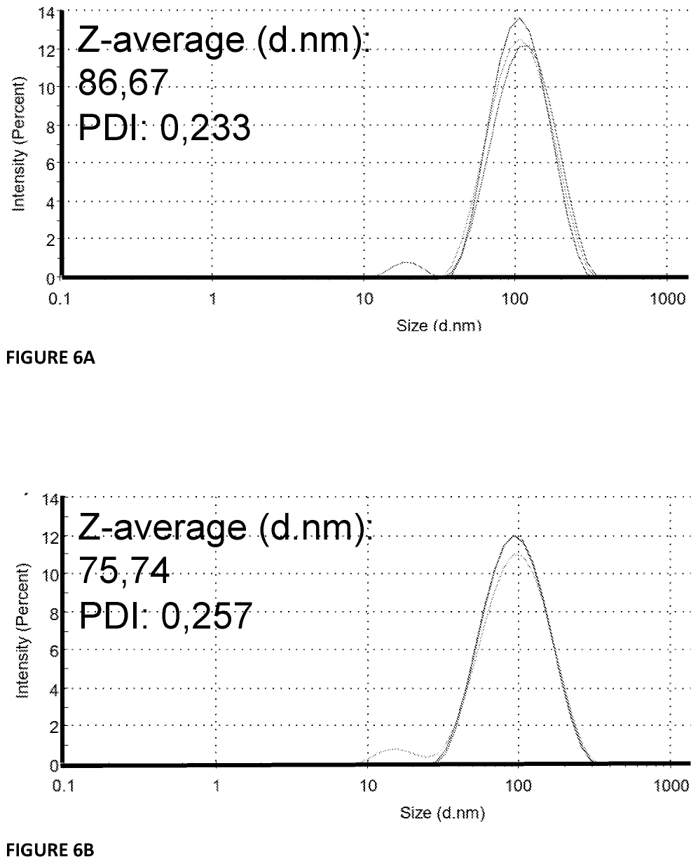

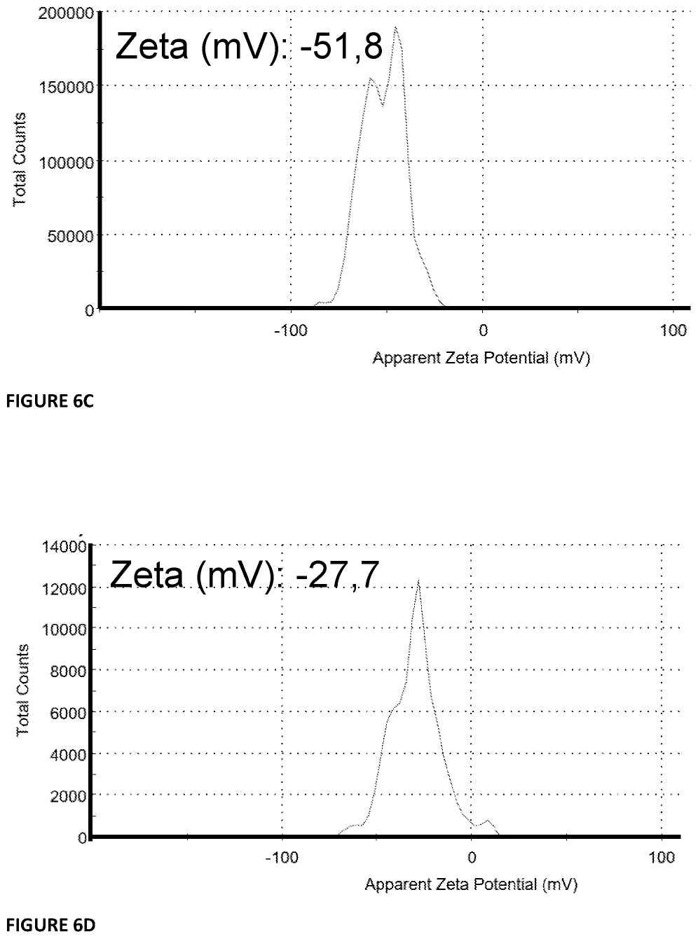

Briefly, 10 mg HAuCl₄.3H₂O (0.025 mM, 1 equivalent) were dissolved in 10 ml distilled water and refluxed for 2 hours. In another flask, 3 equivalents of HP-Dopa-DTPH were dissolved in 30 ml distilled water and pH of the solution was adjusted to 11 using 1 N NaOH. The refluxed solution was then cooled to room temperature, and subsequently the heparin solution was added and stirred overnight at 40° C. The initiation of nanoparticle formation was observed within 30 minutes of addition of heparin solution as the solution turned purple in color. After the overnight reaction, the solution was dialyzed (membrane MWCO: 3.5 kDa) against distilled water for 24 hours to remove any unreacted metal ions and other chemicals from the system. Then the solution was lyophilized to obtain heparin coated gold nanoparticles (HP—Au nanoparticles) as violet fluffy powder. The heparin coated gold particles displayed a hydrodynamic size of 86.67 nm (FIG. 6A) with a zeta potential of −51.8 mV (FIG. 6C)

Finally, Gd³⁺ ions were complexed to the HP Au nanoparticle system utilizing the abundant carboxylates, sulfates and phenolic moieties of dopamine functionalized to the heparin backbone, yielding stable HP—Au—Gd nanoparticles. Briefly, 94 mg HP—Au NPs were dispersed in 90 ml deionized water. The pH of the solution was adjusted to 11 using 1 N NaOH. Gadolinium chloride hexahydrate (19 mg, 20 wt % of HP—Au NPs) was separately dissolved in 4 ml deionized water and dropwise added to the previous solution and allowed to stir overnight. The solution was subsequently dialyzed (membrane MWCO: 3.5 KDa) against distilled water for 24 hours to remove any unreacted metal ions from the system. The purified reaction mixture was lyophilized to obtain HP—Au—Gd NPs as violet fluffy powder. The Gd complexed heparin gold particles displayed a hydrodynamic size of 75.74 nm (FIG. 6B) with a zeta potential of −27.7 mV (FIG. 6D). An inductively coupled plasma mass spectrometry (ICP-MS, Thermo Scientific iCAP™ RQ) analysis of the obtained Gd complexed heparin coated gold nanoparticles was performed. The results of the ICP-MS, normalized to the total amount of materials, are shown in Table 1.

TABLE 1

| | Sample conc. | Dilution | Fe | Gd | Fe | Gd |
|---|---|---|---|---|---|---|
| Sample | (mg/ml) | factor | (μg/l) | (μg/l) | (wt %) | (wt %) |
| HP-Au-Gd NPs | 1 | 1000 | 0.3 | 82.5 | 0.0 | 8.3 |

Relaxivity Measurement

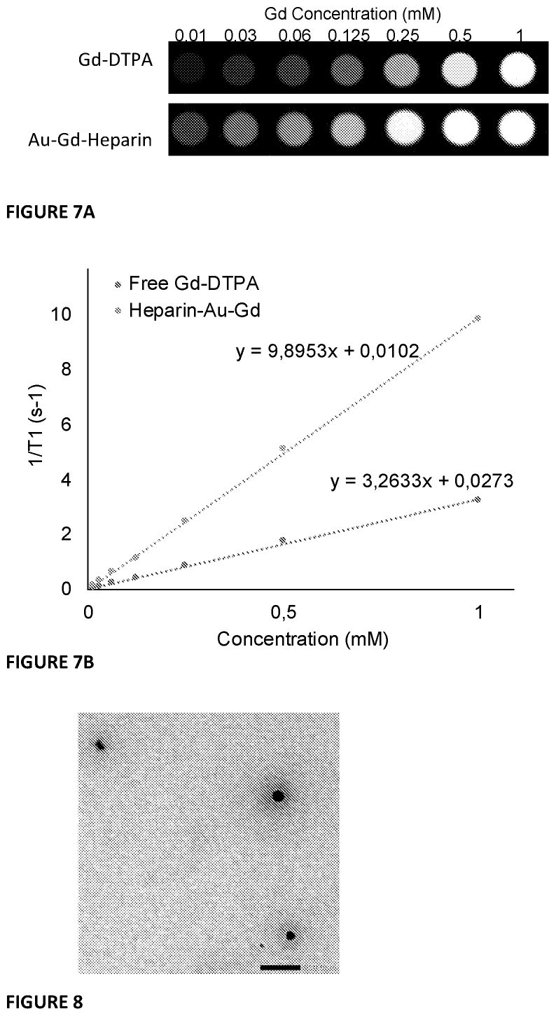

A relaxivity study was performed by evaluating the longitudinal relaxation time T1 of HP—Au—Gd nanoparticles at different Gd concentrations at room temperature by using a 1.0 T-MRI scanner (ICON, Bruker Biospin, Ettlingen, Germany). The commercially available gadolinium contrast agent (Gadopentetic acid or Gd-DTPA) was used as the positive control. The contrast agents were initially placed into syringes and set in the center of the volume coil. The sample temperature was maintained at room temperature. By using the MRI scanner, horizontal single-slice T1-weighted MR images were acquired (spin echo, TR/TE=400/10 ms, slice thickness=2.0 mm, matrix=256×256, field of view (FOV)=38.4×38.4 mm, number of averages (NA)=10, number of slices=1). Horizontal single-slice inversion-recovery MRI was done to calculate the T1 and r1 by using rapid acquisition with relaxation enhancement (RARE) acquisition (TR=20,000 ms, TE=17 ms, inversion time=45, 100, 200, 400, 800, 1600, 3200, 6400, 8000, 10000, 12000 ms, matrix size=64×64, FOV=38.4×38.4 mm, slice thickness=2.0 mm, RARE factor=4, and NA=1). Then, the r1 relaxivities were calculated by using the following equation: r1=(1/T1-1/T1(0))/[Gd] where [Gd] is the concentration of Gd, and 1/T1(0) and 1/T1 are the longitudinal relaxation rate without and with paramagnetic species, respectively. A concentration dependent enhancement in the contrast was observed with both HP—Au—Gd nanoparticles and HP-DTPA (FIG. 7A). Interestingly, the relaxivity of HP—Au—Gd nanoparticles were 9.89 mM⁻¹s⁻¹ which is ˜3 fold higher than that of commercially used Gd-DTPA (3.26 mM⁻¹s⁻¹) (FIG. 7B)

Transmission Electron Microscopy

The morphology of HP—Au—Gd nanoparticles was observed by transmission electron microscopy (TEM). The samples were stained by uranyl acetate (10 μL of a 2% w/v solution) for 30 s and set on carbon-coated 400 mesh Cu grids (Nisshin EM). The grids were dried and transferred to an H7000 TEM (Hitachi Ltd, Tokyo, Japan) for imaging at an acceleration voltage of 75 kV. The HP—Au—Gd nanoparticles showed a particle size of ˜50-60 nm with a heparin corona of ˜10 nm (FIG. 8)

### Example 5

Heparin Coated Gold Nanoparticles for siRNA Delivery

Heparin coated gold nanoparticles (HP-GA-Au-NPs) were prepared using unfractionated heparin conjugated with hydrazide modified gallic acid and bis-aminooxy molecules (FIG. 9). The gallic acid moiety grafted on heparin surface act as a reducing agent to generate gold particles. The gallic acid and the aminooxy linker functionalized heparin were synthesized by conjugating hydrazide modified gallic add and bis-aminooxy linker to the carboxylate residues of heparin (HP) (12% and 21% respectively with respect to the carboxylate groups) following carbodiimide chemistry. The aminooxy derivative was used for conjugating aldehyde terminated siRNA molecules.

Briefly, HAuCl₄.3H₂O (10 mg, 0.025 mM, 1 equivalent) was dissolved in 10 ml distilled water and refluxed for 2 hours. In another beaker, HP-GA-aminoxy (45.5 mg, 0.075 mM, 3 equivalents) was dissolved in 30 ml distilled water and pH of the solution was adjusted to 11 using 1 N NaOH. The refluxed solution was then cooled to room temperature, and subsequently the heparin solution was added to it and allowed to react overnight at 40° C. under constant magnetic stirring. The initiation of nanoparticle formation was observed immediately after the addition of heparin solution. The solution was next dialyzed (membrane MWCO: 3.5 KDa) against distilled water for 24 hours to remove any unreacted metal ions and other chemicals from the system. Then the solution was lyophilized to obtain HP-GA-Au-NPs as violet fluffy powder.

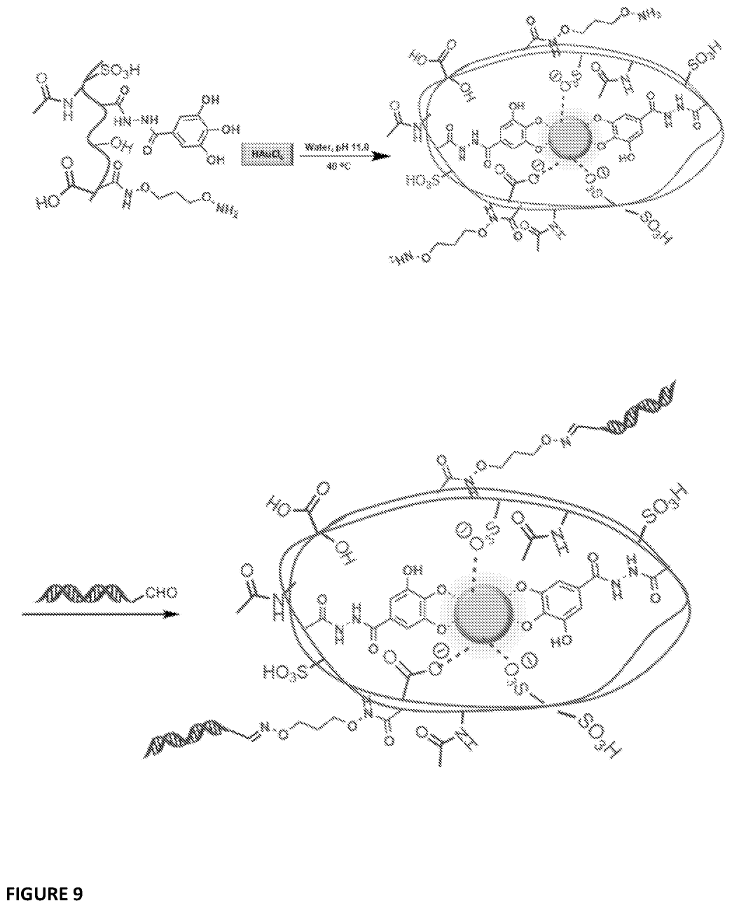

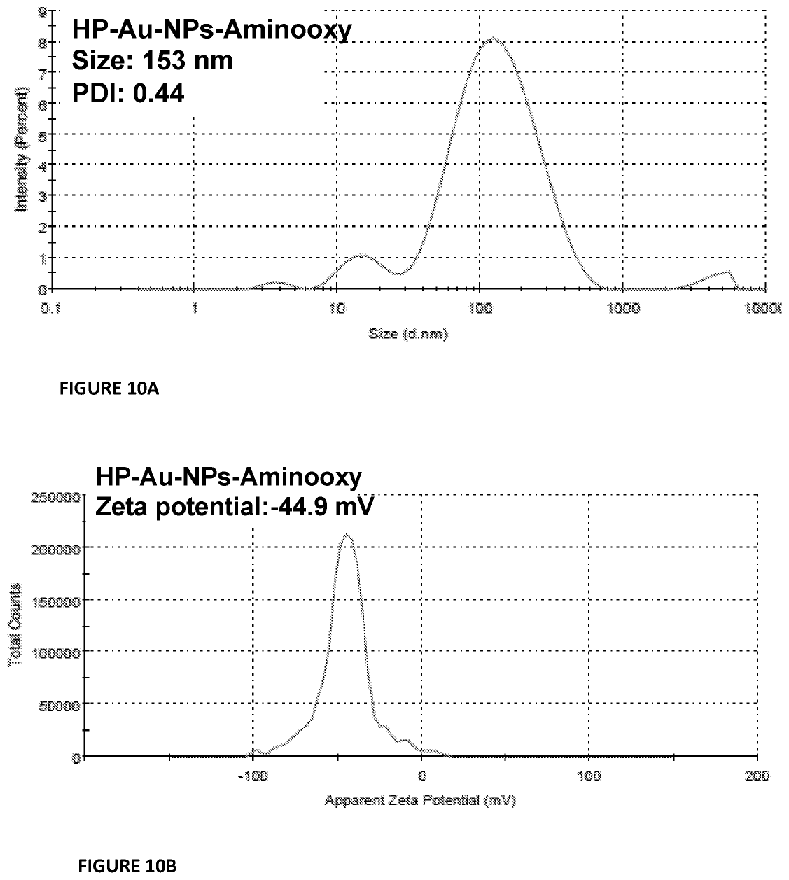

The heparin coated gold particles displayed a hydrodynamic size of 153 nm (FIG. 10A) and a zeta potential of −44.9 mV (FIG. 10B)

Aldehyde functional sense strand and antisense strand of siRNA were synthesized on automated solid phase synthesizer using employing standard synthesis cycle for RNA. Further, both the strands were deprotected, purified by PAGE and the equimolar amount was mixed to form a duplex. To the solution of duplex RNA (5 nL, 0.5 nmol), Au—NP (2.5 μL 2.5 nmol aminooxy, 1 mM aminooxy stock) were added. The reaction mixture was incubated at 25° C. for 48 h. Then the reaction mixture was directly used for conjugation analysis and gene knockdown experiments.

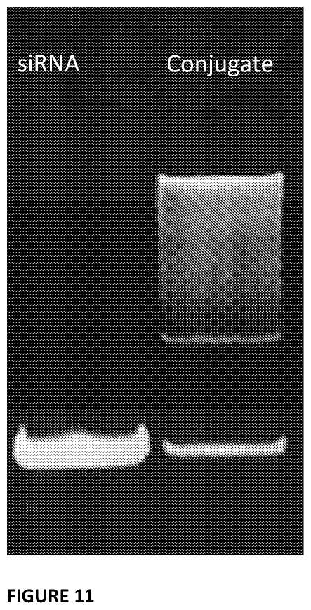

To perform conjugation reaction, the aldehyde modified sense strand in the duplex siRNA (1 eq) was conjugated to the HP-GA-Au-NPs (5 eq) in saline, at room temperature for 48 h. The native PAGE (15%) showed around ˜70-80% of conjugation efficiency (FIG. 11). Conjugated siRNA was used directly for gene knockdown experiments.

### Example 6

Heparin Coated Gold Nanoparticles Labelled with ¹⁸F-Fluorodeoxyribose

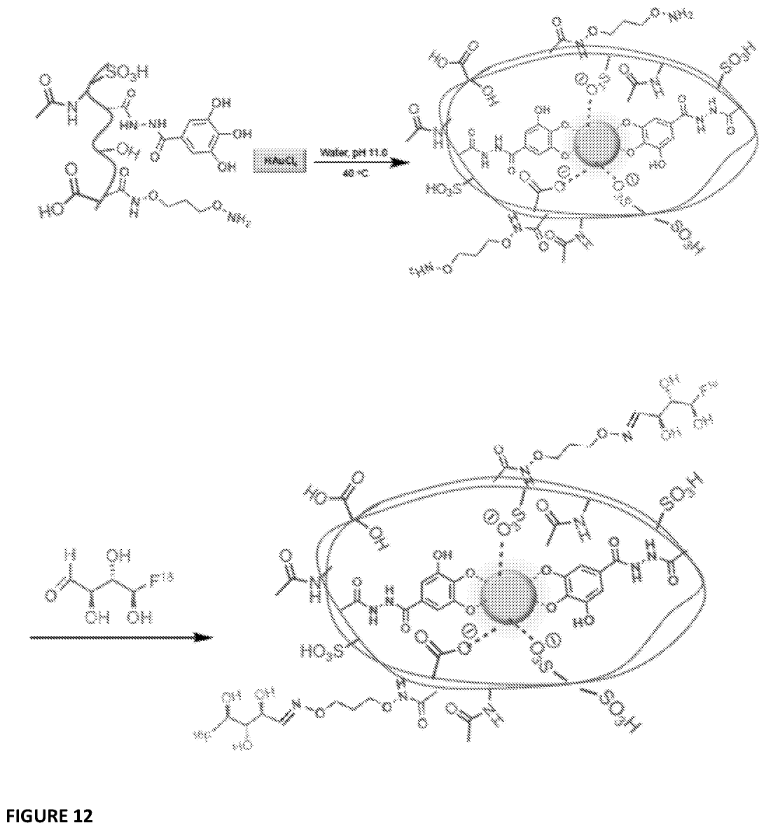

The HP-GA-Au-NPs designed for siRNA delivery are also used for ¹⁸F imaging applications. The lyophilized HP-GA-Au-NPs are conjugated with the radioactive ¹⁸F-fluorodeoxyribose molecules utilizing the free aminooxy moieties present in heparin-Au nanoparticle system following oxime chemistry for PET imaging (FIG. 12). The ¹⁸F labelled HP-GA-Au-NPs are washed and centrifuged at 25,000 RPM to remove unreacted radioactive label and then are injected through the tail vein. Biodistribution studies are performed within 3 h post injection.

### Example 7

Heparin Coated Gold Nanoparticles Labelled with ⁶⁸Ga

Heparin coated gold nanoparticles presented in Example 4 without Gd³⁺ complexation was used to for radiolabeling ([⁶⁸Ga]HP-NPs). ⁶⁸Ga (t_{1/2}=68 min, β+=89% and EC=11%) was available from a ⁶⁸Ge/⁶⁸Ga-generator (Cyclotron Co., Obninsk, Russia) where the ⁶⁸Ge (t_{1/2}=270.8 d) was fixed to a sorbent matrix based on modified titanium dioxide. The nominal ⁶⁸Ge activity loaded onto the generator column was 1850 MBq (50 mCi). The ⁶⁸Ga was eluted with 5 mL of 0.1 M hydrochloric acid. The elution profile was determined by fractionating and measuring the ⁶⁸Ga activity in each successive fraction of the eluate. The pH of the high activity ⁶⁸Ge/⁶⁸Ga-generator eluate (600 μL, 224 MBq) used for reaction was adjusted to pH ˜4.0 by adding aqueous sodium acetate (140 μL, 1 M, pH 4.0) and NaOH (60 μL, 1 M). Then heparin coated gold nanoparticles (Example 4) 2.0 mg (16.67 mM) were added as a solution in 200 μL of 0.1 M aqueous NaOAc (pH 4.0). The reaction mixture was heated to 70° C. in heating block for 15 min.

The reaction mixture (1000 μL) was purified in parallel in 2×500 μL batches by gel filtration on NAP 5 column (cutoff 5 kDa, equilibrated with 2 mL PBS). The columns were first loaded with the reaction mixture (500 μL) and then eluted with PBS (250, 750 and 1000 μL fractions, see Table 2). The load volume and first 250 μL contained no radioactivity and were discarded. The subsequent 750 μL fraction containing the majority of the ⁶⁸Ga—NP was collected. Finally, the small molecule fraction was eluted with 1000 μL. The fractions containing ⁶⁸Ga—NP were combined (92 MBq in 2×750 μL PBS, 46% radiochemical yield (RCY) (decay corrected) from reaction mixture, 18% RCY (decay corrected) from total eluted activity from the generator, in approx. 25 min after eluting the generator).

TABLE 2

| | Radioactivity (MBq) | % RCY (decay corrected) |
|---|---|---|
| Reaction mixture | | 100 |
| Left in reactor | | 0.3 |
| NAP5 load 500 μL | 0 | 0 |
| Fr 1 250 μL | 0 | 0 |
| Fr 2 750 μL (⁶⁸Ga-NP) | 92 | 45.9 |
| Fr 3 1000 μL (LMW fraction) | 78 | 41.8 |
| Left in NAP5 | 20.8 | 11.0 |
| | | 99.1 (recovery) |

Biological Assays

Factor Xa Assay

One of the serious side effects of heparin in clinic is the risk of bleeding. The anticoagulation activity of unfractionated heparin and heparin nanoparticles (HP-NPs) was tested in the factor Xa assay using non-anticoagulated human whole blood.

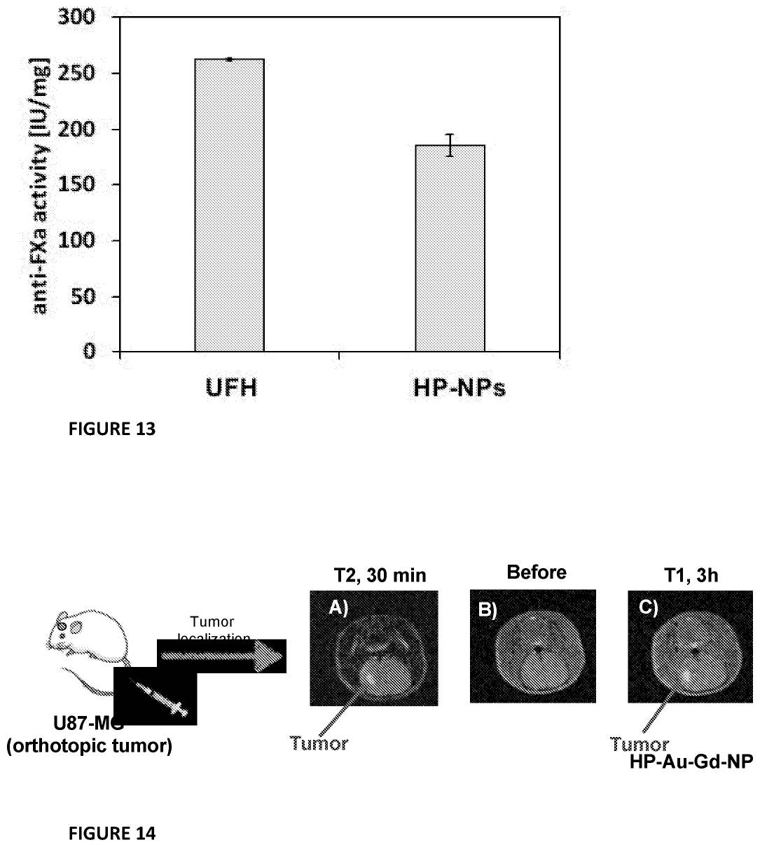

Heparin activity was evaluated by measuring factor Xa activity (BIOPHEN HEPARIN (AT+)) according to the company's protocol. Heparin-FTSC micelles (HP-NPs) shown in Example 1 and unfractionated heparin were diluted with PBS to 5 μg/mL. Each well of 96 well plate was filled with either HP-NPs or unfractionated heparin samples (15 μL). Next, antithrombin solution (from human, 15 μL) was added to each well and was well mixed, and the substrate of FXa (Sxa-11, 75 μL) was added into each well and incubated for 120 seconds. Then, a solution of factor Xa (from bovine, 75 μL) was added into each well and incubated 90 seconds. Finally, 20 mg/mL citric acid solution (100 μL) was added to terminate the reaction. The absorbance at 405 nm of each well was detected with plate-leader. The entire reaction was performed at room temperature. Three different concentrations of heparin were used for the standard curve. A 30% decrease in anticoagulation activity of HP-NPs relative to unfractionated heparin was observed (FIG. 13)

Patient-Derived Cells

The glioma patient samples were obtained from surgeries (Kuopio University Hospital, Finland) during years 2010-2011. BT11 cells were from a glioblastoma-containing oligo-components, BT12 and BT13 cells were from glioblastoma patients, BT11 have oligo-components.

Cell Culture

The patient-derived brain tumour cells (BT11, BT12, BT13) (Le Joncour, 2019) were maintained for up to 20 passages in Dulbecco's Modified Eagle Medium with Nutrient Mixture F-12 (DMEM/F12, Gibco) supplemented with 2 mM L-glutamine, 2% B27-supplement (Gibco), 100 U/ml penicillin and 100 μg/ml streptomycin, 0.01 μg/ml recombinant human fibroblast growth factor-basic (FGF-b, Peprotech), 0.02 μg/ml recombinant human epidermal growth factor (EGF, Peprotech) and 15 mM HEPES-buffer. All the cells were maintained and grown at 37° C. in a humidified atmosphere containing 5% CO₂ unless stated otherwise. Mycoplasma contamination was routinely checked twice a month using the mycoplasma detection kit (11-1050, Minerva Labs).

Human glioma cell line U87MG and murine brain microvessel endothelial cells bEND3 (ATCC) were maintained for not more than 10 and 15 passages, respectively, in DMEM containing 1 g/I glucose, and with 10% FBS, 1% L-glutamine, 100 U/ml penicillin and 100 μg/ml streptomycin. The immortalized human umbilical vein endothelial cells (HuAR2T) were obtained from the Ojala Lab (University of Helsinki, Finland) and maintained from passages 15 to 25 in Endothelial Basal Medium 2 (C-22211) added with Supplements Pack Endothelial Cell GM2 (C-39211) (Promocell) and 2 μg/ml doxycycline.

Animal Experiments

Animal experiments were approved by the Committee for Animal Experiments of the District of Southern Finland (ESAVI/6285/04.10.07/2014). Eight- to twelve-week old male FVB/NRj (Lab Animal Center, Helsinki, Finland) mice (n=3) were injected in the tail vein with 100 μg (200 μl, PBS) nanoparticles or vehicle. Animals were euthanized at 1 or 24 hrs post-injection, brains and control organs were collected and snap-frozen upon tissue processing and nanoparticle-related fluorescence quantification.

Six-week old immunocompromised NMRI-nu (Rj:NMRI-Foxn1^{nu}/Foxn1^{nu}, Janvier Labs) mice were intracranially engrafted using a stereotaxic injector (World Precision Instruments) in the corpus callosum with 10⁵BT12 cells in 5 μl of PBS under 3% isoflurane anesthesia. Post-operative painkiller (Temgesic®) was locally administered for two days. 12 days after tumour implantation, animals were randomized and nanoparticles (5 mg/kg in PBS, intravenously, n=6) or doxorubicin (1.5 mg/kg in PBS, intravenously, n=6) was injected every three days for a duration of 12 days or until the physical manifestation of tumour burden. Control animals (n=3) were treated under the same modalities with the vehicle (PBS, 200 μl). At the endpoint of the experiment, animals were euthanized and brains were snap-frozen in −50° C. isopentane (Honeywell) until tissue processing as described above.

In Vivo MRI Imaging

Female BALB/c mice (6-weeks-old) were inoculated intracranially with U87-MG cells (1×104 cells). After 15 days, the animals were studied by MRI after intravenous injection of HP—Au—Gd nanoparticles at a dose of 10 mg/kg based on Gd. The MRI measurements were done in 1T MRI equipment (Bruker BioSpin) with the following imaging parameters: spin-echo method, TR=400 ms, TE=12 ms, FOV=30×30 mm, matrix size=188×188, and slice thickness=1.8 mm. Images were acquired at defined time points. The mouse was anesthetized and exact location and volume of glioblastoma cells in the brain was quantified by T2 contrast after 30 min. A time dependent enhancement in contrast in the brain was observed (FIG. 14A-C). Efficient localization of HP—Au—Gd-NPs at the glioma after 3 h of injection was observed by the T2 relaxation, displaying efficient glioma targeting following an unknown mechanism.

MTT-Proliferation Assay

5000 cells/well were plated on 96-well plates in 3-10 replicates (100 μl volume). At the indicated time points, 10 μl of 3-(4,5-dimethylthiazol-2-yl)-2,5-diphenyltetrazolium bromide (MTT) (5 mg/ml in PBS) was added and the cells were incubated for 2 hours. Finally, the cells were lysed (10% SDS, 10 mM HCl) o/n and the absorbance was measured at 540 nm using Multiskan Ascent software version 2.6 (Thermo Labsystems).

Immunofluorescence

In order to attach the suspension cells, glass coverslips were first coated with Poly-D-Lysin (Sigma) according to manufacturer's instructions. Cells were cultured for 24 h after which they were fixed with 4% PFA and permeabilized with 0.5% NP40 in PBS. The cells were blocked with 3% BSA-PBS and incubated with the primary and fluorescently labelled secondary antibodies (Alexa Fluor®, Life technologies). Nuclei were visualized with 4′,6-diamidino-2-phenylindole (DAPI) (Vectashield®, Vector Laboratories).

Isolation of Patient-Derived Cells

Tumors were collected into solution (0.1% BSA (Biowest), 1M glucose in PBS) centrifuged for 1 min at 250×g and suspended into trypsin-hyaluronidase-kynurenic acid-mixture (1.33% trypsin, 0.7% hyaluronidase, 0.2% kynurenic acid, 10 000 U/ml DNase in 1×HBSS-solution containing 1M glucose and 1M HEPES). Samples were incubated at +37° C. for 15-30 min and triturated with a pipette to break down tissues clumps. After filtering through 100 μm nylon mesh filter the cells were centrifuged for 5 min at 250× g. Cell pellets were suspended into sucrose solution (30.8% glucose in 0.5×HBSS) and centrifuged again for 10 min at 500× g. Cells were suspended in 2 ml of 1×EBSS-medium and pipetted on top of 15 ml of BSA-EBSS-HEPES-solution (4% BSA, 1M HEPES in 1×EBSS). After centrifugation for 7 min at 350× g, the cells were suspended into the complete growth medium. Trypsin, hyaluronidase, kynurenic acid and DNase were obtained from Sigma. HBSS and EBSS were obtained from Gibco and 1M HEPES-buffer from Lonza.

Animal Tissue Processing and Analysis

Snap-frozen xenografted brains were cut using a cryotome (CryoStar™ NX70, Thermo Scientific) into series of 9 μm-thick coronal sections collected from the frontal to the anteroposterior part of the tumor. Tissue sections were fixed in 4% PFA, blocked with 5% FBS and 0.03% Triton X (Sigma) and stained for the human vimentin and mouse endothelial podocalyxin by immunofluorescence. Whole brain sections were then scanned using a slide scanner (3DHistec). When required, verification of co-located staining was performed on 9 μm-distant consecutive sections of each brain. Primary tumor volume determination was obtained by measuring the tumor area of 100 μm-distant consecutive sections covering the whole tumor (up to 30 histologic sections). Quantification of the different tumor parameters (tumor cell invasion, number of angiotropic satellites) was performed on 10 sections equally distributed along the whole tumor.

Microscope Imaging

The samples were imaged with a fluorescent upright microscope (Zeiss Axioplan and Axioimager) using AxioVision software. For the light microscopy, the cells were visualized and imaged with an inverted epifluorescence microscope (Zeiss Axiovert 200) equipped with AxioVision software. Confocal images were taken with Zeiss LSM 780 and 880 equipped with appropriate lasers.

In Vitro Blood-Brain Barrier Model and Permeability Measurements

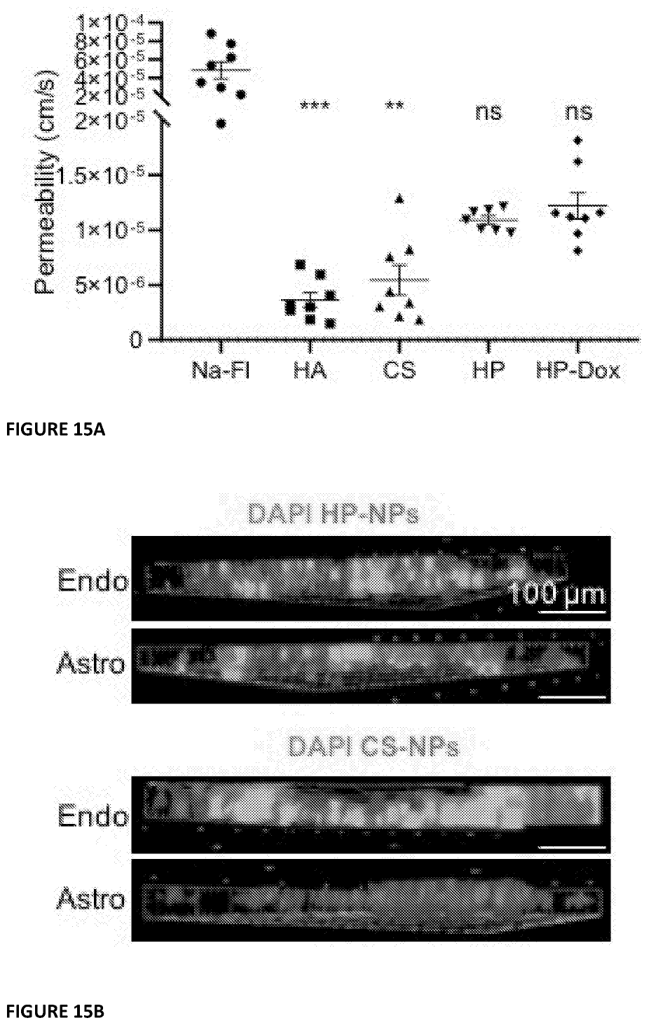

An in vitro blood-brain barrier assay was established as previously described (Le Joncour V, et al., J Vis Exp. 2019(146)). It consists of a multicellular in-vitro model of BBB that comprises an endothelial cell (EC) layer on one side and astrocyte layer on the other side of a Transwell® membrane insert. The permeability of this multicellular barrier is very close to that of the real BBB. The test samples, comprising hyaluronic acid nanoparticles, (HA-NPs), chondrotin-sulfate nanoparticles (CS-NPs), the heparin nanoparticles (HP-NPs) of Example 1, and doxorubicin loaded heparin nanoparticles (HP-Dox-NPs) were incubated in 1 mg/mL concentration for 24 h on the blood side (endothelial cells in the upper chamber) and their transfer to the brain side was followed (astrocytes below the membrane and the lower chamber). Passage of nanoparticles was measured by fluorescence measurements following diffusion over time from the blood to the brain side of the inserts. 100 μl samples suspended in media were collected from both the blood and the brain side of the inserts at indicated timepoints and transferred to 96-well plate for fluorescence measurements. Results of 6 experimental replicates were quantified with plate reader with filter set on 480/560 nm. Fluorescein molecules were used as control to assess the integrity of BBB and for studying the diffusion parameters. Only heparin nanoparticles (HP-NPs and HP-Dox-NPs) displayed permeability of BBB in the artificial in-vitro BBB model (FIG. 15).

In Vivo Blood-Brain Barrier Permeability in Healthy Mice

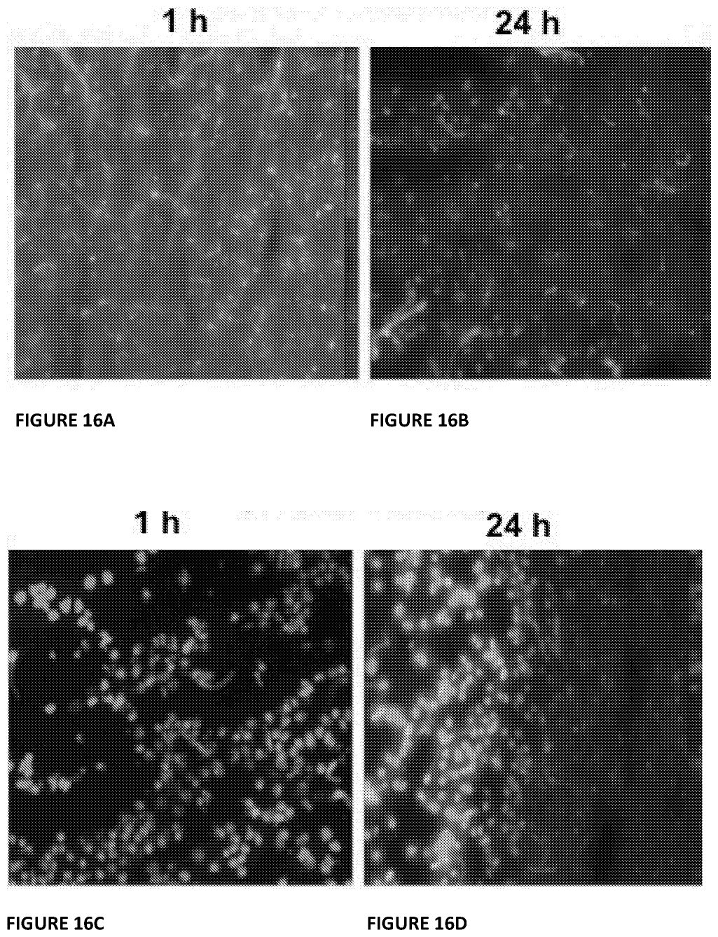

The in-vivo BBB permeability study was carried out in immunocompetent FVB/NRj mice. Briefly, adult male immunocompetent FVB/NRj mice were given caudal vein infusions (100 μg in 200 μl of PBS) of chondrotin-sulfate nanoparticles (obtained by conjugating FTSC to CS polymer as presented in Example 1) (CS-NPs), heparin nanoparticles (HP-NPs) (Example 1). Control animals were injected with 200 μl of vehicle (PBS). Brain tissue and selected organs (kidney, liver and lungs) were then collected after 1 h (n=3) and 24 h (n=3) in order to verify the delivery and brain homing over time (FIG. 16A-D). Only low levels of fluorescence related to intraparenchymal diffusion of CS-NPs (green) were detected in the brain (blue), outside the blood vessel capillaries (red) after 1 h (FIG. 16C). Nanoparticle-related signal was undetectable in brain or vasculature after 24 h (FIG. 16D). On the other hand, high levels of HP-NPs fluorescence (green) were detected in the brain parenchyma (blue), outside the blood vessel capillaries (red) 1 h post-injection (FIG. 16A). These observations suggested the passage of the HP—NP through the blood-brain-barrier in vivo.

In-Vivo Glioma Study by the Intracranial Xenograft Model

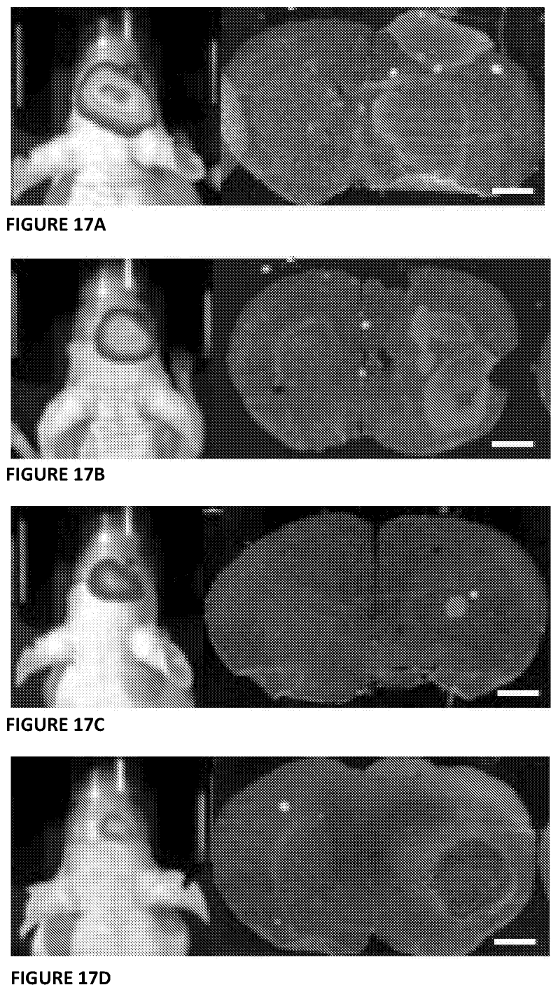

Patient-derived BT12 expressing the luciferase reporter protein were intracranially implanted into the corpus callosum of immunocompromised nude mice, to provide an animal model of glioblastoma progression. Unlike serum-grown cell lines growing in the brain as a cyst, BT12 cells expand in the mouse brain by forming large primary tumor mass surrounded by highly diffusive invasive glioblastoma cells co-opting to brain blood vessels. Luciferase reporter protein expressed by BT12 cells allow the live bioluminescence imaging of the tumors over the course of the experiment. 12 days after tumor engraftment and confirmation of homogenous tumor growth within the cohorts, heparin nanoparticles (Example 1), (5 mg/kg) doxorubicin loaded heparin nanoparticles (5 mg/kg, contain 4% DOX by weight), doxorubicin (1.5 mg/kg) or vehicle (PBS, 200 μl) were infused in the tail vein of the tumor-bearing mice every 3 days for 12 additional days. Bioluminescence imaging and immunofluorescence micrograpy of the brain were performed at day 24 (FIGS. 17A-D). Vehicle-treated animals showed the classical tumor development previsouly described for BT12 glioma, i.e. several large tumor masses distributed within the white and gray matter in addition to large trails of invasive cells classically using the pontine encephalic connection to access the contralateral hemisphere (FIG. 17A). Doxorubicin-treated animals exhibited a similar tumor growth (FIG. 17B) with additional necrotic features in the primary tumor mass indicating cytotoxic features on the proliferative tumor core. Strikingly, the animals treated with plain heparin nanoparticles (HP—NP) showed the greatest tumor reduction (FIG. 17C) with no visible signs of tumor invasion and nearly complete disappearence of the primary tumor mass. The anti-tumor properties of the heparin nanoparticles conjugated to doxorubicin (HP-Dox-NP) exhibited less pronounced anti-tumor properties than HP—NP alone, but kept the tumor-toxic effect of the plain doxorubicin confirmed by the presence of necrotic features in the primary tumor mass (FIG. 17D).

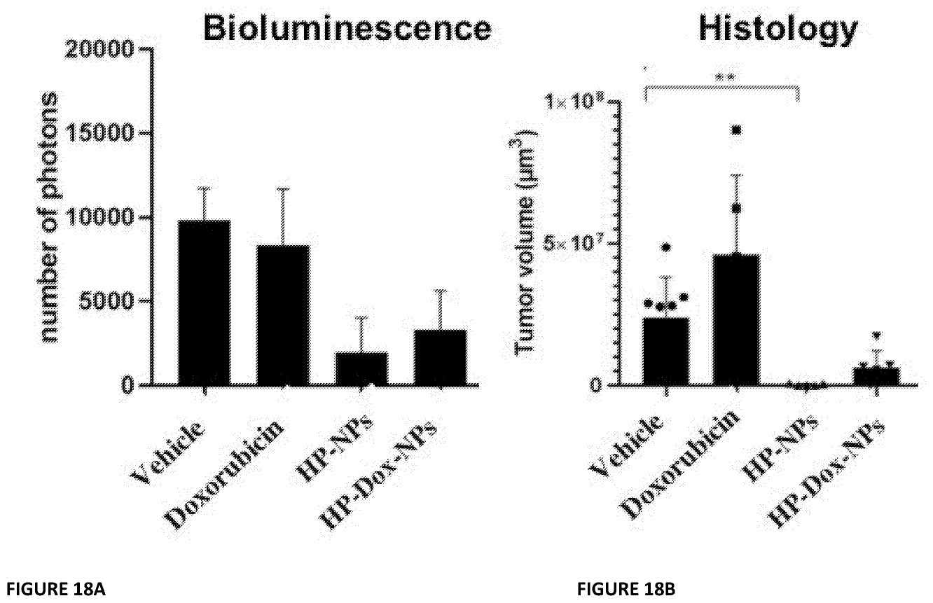

The quantification of tumor volume was performed by measuring the bioluminescence (FIG. 18A), and by histology (FIG. 18B), showing the excellent brain tumor reduction obtained by the heparin nanoparticles of the invention, and—surprisingly— showing that the tumor reduction efficacy was even better for the unmodidifed nanoparticles than for the nanoparticles modified by the anti-cancer agent doxorubicin.

In Vivo Study of Reduction of Expression of Heparin Binding EGF-Like Growth Factor in Mouse

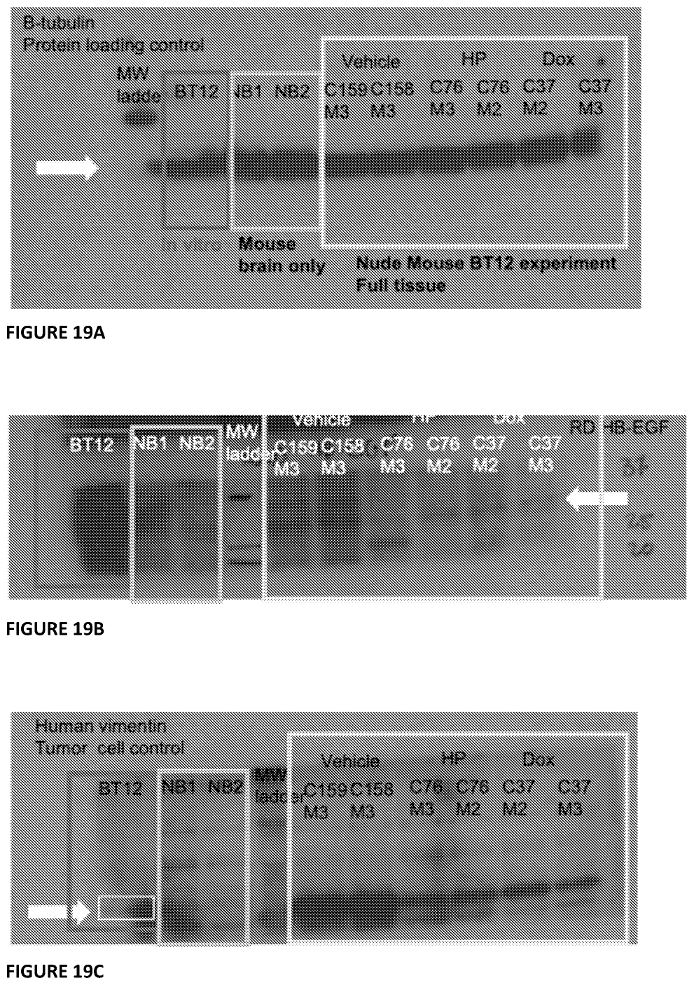

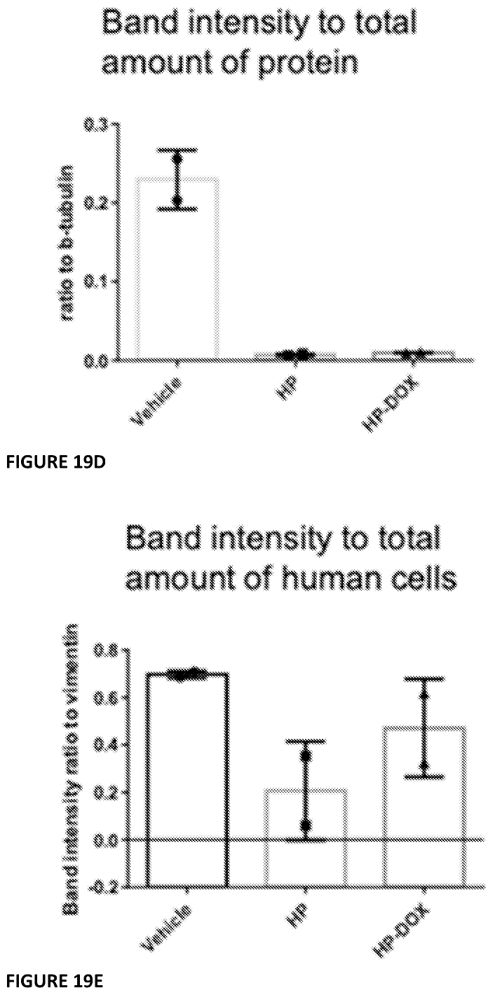

Heparin binding EGF-like growth factor (HB-EGF) plays a key role in gliomagenesis (Shin, 2017) and development of temozolomide resistance by overexpression of HB-EGF (Séry, 2017). Therefore, HB-EGF is a possible target in the suppression of glioma by HP-NPs. The present inventors showed that HB-EGF expression was significantly reduced in HP—NP treated PDX mouse compared to the HP-DOX-NPs or untreated control, cf, western blot data given below and FIGS. 19A-D.

Western Blot

The cells or brain samples were lysed either in RIPA-buffer (150 mM sodium chloride, 50 mM Tris, 2 mM EDTA, 0.5% sodium deoxycholate, 0.1% sodium dodecyl sulfate SDS, 0.5 mM DTT, Complete ultratablet protease inhibitor, (Roche)) or in SDS-buffer (150 mM Tris, pH 6.8, 1.2% SDS, 30% glycerol, 15% β-Mercaptoethanol). Protein concentrations of the extracts were determined spectrophotometrically (Pierce BCA Protein Assay Kit, Thermo Scientific). 10-20 μg of protein were loaded to a 12% SDS-PAGE gel followed by a transfer to PVDF membrane (Immobilon, Millipore) either by electroblotting in transfer buffer (20% MetOH, 250 mM Tris, 1.9 M glycine) 100V for 1 h at +4° C. or by Transblot Turbo device (Biorad) using manufacturer's instructions. After blocking (5% BSA in 0.1% TBS-Tween) the membrane was probed with the primary antibodies o/n at +4° C. After several washes, the membrane was probed with horseradish peroxidase-conjugated secondary antibodies, washed again and finally visualized using the SuperSignal West Pico kit (Thermo Scientific) or with Clarity Western ECL (Bio-Rad).

Quantification and Analyses

Band intensities (arbitrary pixel intensity values) were determined in ImageJ and ratio were calculated as (band intensity protein of interest)/(band intensity of loading control) with either B-tubulin or human vimentin used as loading controls. Bar plots were obtained from a representative dataset using the GraphPad Prism 8 software (La Jolla, USA) using two-tailed, nonparametric Mann-Whitney U test. The experiment was repeated twice with duplicates of each conditions.

Competitive In Vitro Blocking Assay was Performed in Mouse Brain Endothelial Cell and Human Glioma Cell with Unfractionated Heparin

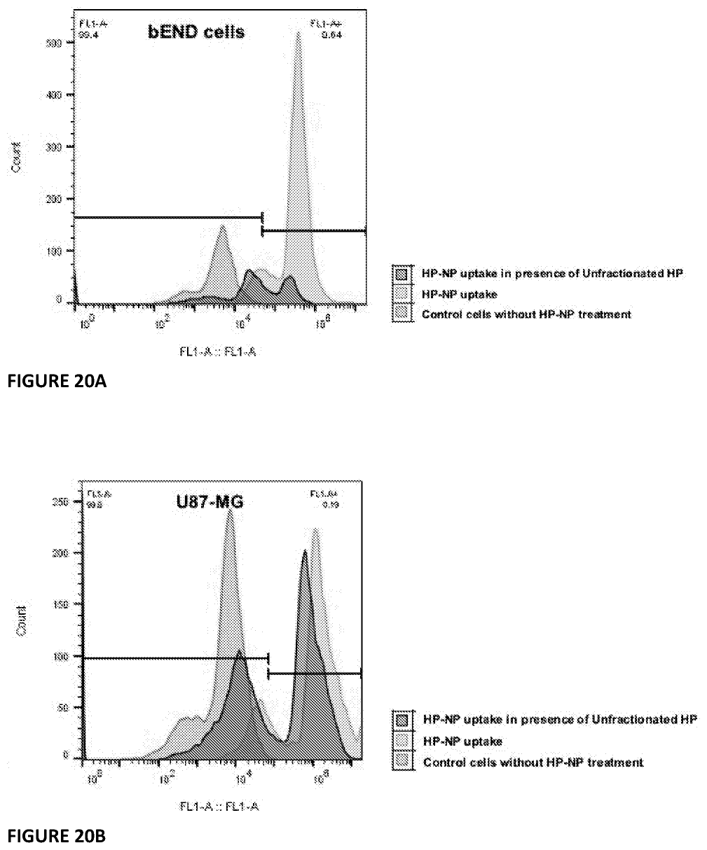

Uptake of HP-NPs by the brain endothelial cells and glioma cells occurs by receptor mediated endocytosis. A competitive blocking assay was performed in mouse brain endothelial cells (bEND3) and human glioma cell line (U87-MG). It was found that pre-treatment of these cells with free unfractionated heparin blocked the cellular uptake of fluorescently tagged HP-NPs by 40% and 24% in bEND3 and U87-MG cells respectively as observed by flow cytometry assay (FIGS. 20A and 20B). This clearly demonstrates that cellular uptake of HP-NPs is regulated by receptor mediated endocytosis.

For the uptake study 50,000 mouse brain endothelial cells (bEND3) or glioma U87-MG cells were plated in a 24-well plate and incubated overnight at 37° C., 5% CO₂ For the competitive blocking study, the cells were pretreated with 10 mg/ml unfractionated heparin sulphate (dissolved in medium) for 1 h and then the medium was removed, and the cells were washed with dPBS. Subsequently, the cells were incubated with HP—Au-NPs conjugated with fluorescein (0.2 mg/ml in cell culture medium) for 2 hr. The cells were then washed 3 times with dPBS and trypsinized to form single cell suspension and were subsequently used for flow cytometry using a BD C6 Accuri flow cytometer.

Rat Plasma Stability of ⁶⁸Ga—HP-NPs (Example 7)

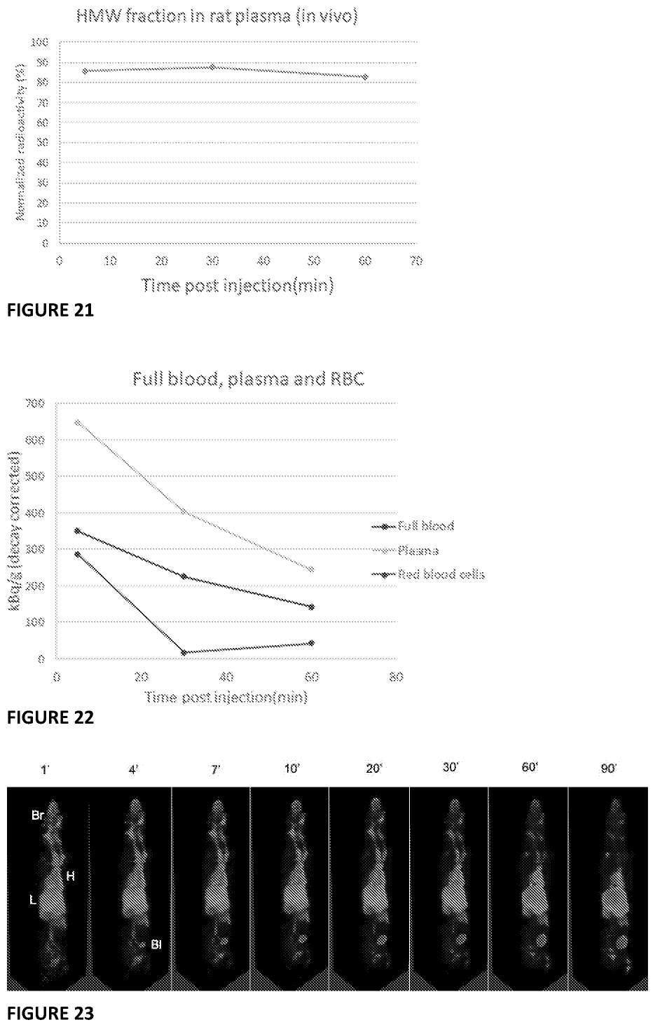

Blood was withdrawn from the tail vein to heparinized Eppendorf tubes at the specified time points and immediately cooled on ice. A small aliquot (approx. 50 μL) of whole blood was weighed and measured for radioactivity in a well counter. The remaining blood was centrifuged (4000 RCF, 4° C., 5 min) to separate red blood cells (RBC) and plasma. Aliquots of plasma (100 μL) and RBC (100 μL) were weighed and measured for radioactivity. The 100 μL plasma sample was diluted with PBS (1400 μL) and then centrifuged in a centrifugal filter (Amicon Ultra 2 mL Ultracel® 10 k regenerated cellulose 10 000 nominal molecular weight limit (NMWL), 4° C.) until the liquid surface reached the rim of the lower rim of the filter. Additional 1400 μL of PBS were added and centrifuged in a similar way once more. The activity in the filtrate was measured which corresponds to low molecular weight (LMW) unbound ⁶⁸Ga while the remaining activity in the filters represent all high molecular weight (HMW) compounds including intact [⁶⁸Ga]HP-NPs (Example 7). All radioactivity data was decay corrected and normalized for the sample weight. The results are illustrated in FIG. 21 and FIG. 22.

Biodistribution of Gallium-68 Radiolabeled Nanoparticles

Sprague Dawley rats, (n=4, male, healthy, weight 267-298 g) were used for in vivo PET/MRI and ex vivo frozen tissue autoradiography assessment of biodistribution, brain targeting and in vivo stability.

A target amount of 20 MBq [⁶⁸Ga]HP-NPs (Example 7) was injected intravenously in rats sedated by sevoflurane, in a volume of around 500 μl PBS. The injected amount corresponded to approximately 0.5 mg of nanoparticle. No animal reacted adversely to the injection.

Two of the rats were examined by PET for 90 minutes from injection using a nanoPET/MRI scanner (Mediso, Hungary). The dynamic whole-body distribution was measured by performing multiple sequential whole body passes across the PET field of view (FOV). After the PET scan, the animals were euthanized by IV sodium thiopental (Apoteket AB, Stockholm, Sweden) and then a Spin-Echo Multi-FOV MRI sequence was performed for anatomical co-registration of PET data. PET/MRI data was analyzed in PMOD 4.0 (PMOD Technologies, Zurich, Switzerland), using the PFUSEIT module. The brain regions of interest were overlaid using the PET/MRI brain images co-registered with the Px Rat (W. Schiffer)-T2 atlas.

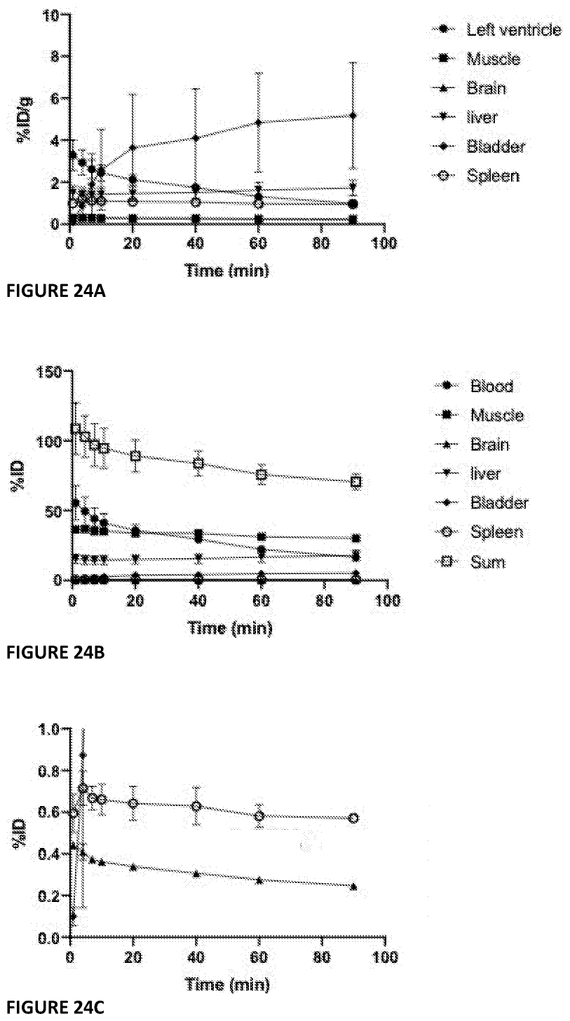

In two of the rats, blood samples were drawn at 5, 30 and 60 minutes after administration, to assess [⁶⁸Ga]HP-NPs (Example 7) partitioning in blood plasma and whole blood over time. After 60 minutes, the rats were euthanized. Tissues (whole brain, liver, spleen) were excised, snap frozen in isopentane chilled with dry ice, mounted in OCT media and processed into 10 μm sections by a cryotome. The sections were then exposed to a phosphorimager plate for 3 h and developed using a Typhoon phosphorimage reader. The sections were then counterstained with hematoxylin/eosin according to standard procedures. The results are illustrated in FIG. 23 and FIGS. 24A-C.

### REFERENCES

Abbott, Cell Mol Neurobiol 25, 5-23 (2005).
Anraku et al., Nature Communications 8, 1001 (2017).
Belhadj, et al., Oncotarget 8, 66889-66900 (2017).
Bhavna et al., Drug Development and Industrial Pharmacy 40, 278-287 (2014).
Borsig, L. in Progress in Molecular Biology and Translational Science, Vol. 93. (ed. L. Zhang) 335-349 (Academic Press, 2010).
Chen et al., Adv Drug Deliver Rev 64, 640-665 (2012).
Cho, K. J. et al., Bioconjugate Chemistry 19, 1346-1351 (2008).
Choi et al., Macromolecular Research, Vol. 19, No. 2, pp 180-188 (2011).
Chung et al., Journal of Controlled Release 143, 374-382 (2010).
Davis, Clin J Oncol Nurs 20, S2-8 (2016).
Di Carlo et al., Neurosurg Rev. (2019); 42(2):263-275
Ding et al., Chinese patent application No. CN106188320A.
Donato et al. Blood 126, 494-499 (2015).
Gayathri et al., Journal of Bionanoscience, Volume 9, Number 6, 2015, pp. 409-423
Godinho et al., Molecular Therapy 26, 2580-2591 (2018).
Han et al., International Journal of Pharmaceutics 313, 181-188 (2006).
Jeon et al., Biomaterials 27 (2006) 1598-1607.
Kemp et al., Wiley Interdiscip. Rev. Nanomed. Nanobiotechnol. 2:77-87, (2010).
Kemp et al., Nanotechnology 20(45), 455104 (2009).
Kuo et al., Materials Science and Engineering C 77 (2017) 680-689.
Le Joncour et al., EMBO Mol Med (2019) 11:e9034
Lin, et al., Oncologist 23, 468-473 (2018).
Lundy, et al., ACS Nano 13, 97-113 (2019).
Ma et al., Thrombosis Research 105, 447-453 (2002).
Markoutsa et al., Pharmaceutical Research 31, 1275-1289 (2014).
Mazur et al., Nanoscale (2013), 5, 2692-2702
Mei et al., ACS Applied Materials & Interfaces 8, 9577-9589 (2016).
Min et al., Pharmaceutics (2010), 2, 119-135.
Mousa et al., Thromb Haemost 102, 258-267 (2009).
Neuhaus, et al., Biomaterials 161, 129-143 (2018).
Patel, et al., CNS Drugs 23, 35-58 (2009).
Pourgholi, et al., Biomed Pharmacother 77, 98-107 (2016).
Qin et al., Chemical and Pharmaceutical Bulletin 55, 1192-1197 (2007).
Ruan et al., ACS Applied Materials & Interfaces 9, 17745-17756 (2017).
Séry et al., Biochemical and Biophysical Research Communications 493 (2017) 1377-1383,
Shaikh et al., Journal of Applied Polymer Science (Volume 70, Issue 1, 1998, pp. 195-201).
She et al., Biomaterials 34, 2252-2264 (2013).
Shergalis et al., Pharmacological Reviews July 2018, 70 (3) 412-445.
Shin, et al., Oncogene (2017) 36, 4610-4618,
Siegel et al., CA Cancer J Clin 69, 7-34 (2019).
Singh, et al., Int. J. Mol. Sci. (2018), 19, 1979
Stalmans, et al., PLOS ONE 10, e0139652 (2015).
Stewart, Cellular and Molecular Neurobiology 20, 149-163 (2000).
van der Wall et al., Journal of Thrombosis and Haemostasis 15, 74-79 (2017).
Wang, et al., Advanced Functional Materials 26, 7873-7885 (2016).
Wang et al., Chinese Journal of Polymer Science Vol. 26, No. 4, (2008), 381-391.
Yang et al., U.S. patent application Ser. No. 12/857,986, Publication No. 2011/0054236 A1 (2011).
Zacharski, et al., Expert Opinion on Investigational Drugs 17, 1029-1037 (2008).
Zhang et al., Biomaterials 33, 5414-5422 (2012).

1. A method for the treatment or diagnosis of a brain disorder, comprising administering to a mammal in need of such treatment or diagnosis a nanoparticle comprising chemically modified heparin, wherein the heparin has been chemically modified by attaching hydrophobic moieties to functional groups of the heparin, said functional groups being selected from hydroxy groups and carboxy groups.

2. The method according to claim 1, wherein said hydrophobic moieties have been attached to said functional groups by allowing said functional groups to react with one or more compounds selected from cholesterol, fluorescein, ceramides, fatty acids, hydrophobic polymers and hydrophobic monomers.

3. The method according to claim 2, wherein at most 10% of the total number of said functional groups are chemically modified.

4. The method according to claim 2, wherein said hydrophobic polymers are selected from poloxamers, poly(lactic-co-glycolic acid), poly-β-benzyl-1-aspartate, poly-γ-benzyl-1-glutamate, and copolymers of poly-β-benzyl-1-aspartate and poly-γ-benzyl-1-glutamate.

5. A method for the treatment or diagnosis of a brain disorder, comprising administering to a mammal in need of such treatment or diagnosis a nanoparticle comprising a core particle of metal or metal oxide having a coating of heparin.

6. The method according to claim 5, wherein the core particle is a gold particle or a superparamagnetic iron oxide particle.

7. The method according to claim 1, wherein the heparin has a molar mass of at least 8 kDa.

8. The method according to claim 1, wherein the nanoparticle has a size in the range of from 40 nm to 200 nm.

9. The method according to claim 1, wherein the nanoparticle is covalently or physically linked with a small molecule drug or a large molecule drug, such as a nucleic acid, an antibody, a peptide or a protein.

10. The method according to claim 1, wherein the brain disorder is a brain tumor, a neuroinflammatory disease, or a cognitive disease.

11. The method according to claim 5, wherein the heparin has a molar mass of at least 8 kDa.

12. The method according to claim 5, wherein the nanoparticle has a size in the range of from 40 nm to 200 nm.

13. The method according to claim 5, wherein the nanoparticle is covalently or physically linked with a small molecule drug or a large molecule drug, such as a nucleic acid, an antibody, a peptide or a protein.

14. The method according to claim 5, wherein the brain disorder is a brain tumor, a neuroinflammatory disease, or a cognitive disease.
